(19) United States
(12) Patent Application Publication
(10) Pub. No.: US 2012/0077274 A1
(43) Pub. Date: Mar. 29, 2012

(54) CHEMICAL OR BIOCHEMICAL ANALYSIS APPARATUS AND METHOD FOR CHEMICAL OR BIOCHEMICAL ANALYSIS

(75) Inventors: Chung-Fan Chiou, Hsinchu City (TW); Jyh-Chern Chen, Taipei County (TW); Ming-Te Lin, Taipei County (TW); Chao-Chi Pan, Hsinchu City (TW); Chein-Shiu Kuo, Taipei City (TW); Jenn-Yeh Fann, Hsinchu County (TW)

(73) Assignee: INDUSTRIAL TECHNOLOGY RESEARCH INSTITUTE, Hsinchu (TW)

(21) Appl. No.: 12/982,659

(22) Filed: Dec. 30, 2010

Related U.S. Application Data
(60) Provisional application No. 61/385,945, filed on Sep. 23, 2010.

(51) Int. Cl. G01N 35/00; C12M 1/34

(52) U.S. Cl. 436/43; 422/63; 435/287.3; 422/66

(57) ABSTRACT

A chemical or biochemical analysis apparatus includes: a computer processor; at least one controller electrically coupled to the computer processor; at least one first base configured with a plurality of dispensing tube assemblies arranged in alignment and electrically coupled to the at least one controller, independently; at least one second base configured with a plurality of the detectors arranged in alignment and electrically coupled to the at least one controller; and a stage, for carrying the at least one multi-well strip having a plurality of wells arranged in alignment and for transporting the multi-well strip to pass through and underneath the plurality of dispensing tube assemblies and the plurality of the detectors arranged in order, electrically coupled to the at least one controller.

### CROSS REFERENCE TO RELATED APPLICATIONS

This application claims the benefit of U.S. Provisional Application No. 61/385,945, filed on Sep. 23, 2010, the entirety of which is incorporated by reference herein.

### BACKGROUND

1. Technical Field

The disclosure relates to an apparatus and method for chemical or biochemical analysis, and in particular relates to an apparatus and method for chemical or biochemical analysis which is capable of dispensing a variety of predetermined amounts of the liquid samples and reagents separately into a plurality of wells, respectively, and performing detection of chemical or biochemical reactions occurring in each of the plurality of wells in one operation.

2. Description of the Related Art

Recently, developments in life science fields have occurred at a breathtaking rate, with great promise in the medical, agricultural and environmental science fields, for the reshaping of the respective fields. Particularly, human genome sequencing breakthroughs in the 1990s, has led to advancements in the genomics, proteomics and metabolomics fields, which are causing unprecedented changes in modern healthcare. Research of the related “omics”, involve the measurement of large quantities of biological molecules, such as genes, proteins, lipids, carbohydrate and metabolites. The success of these efforts depends, in part, on the development of efficient tools that will automate and expedite the testing and analysis of hundreds and thousands of biological materials. For many of the chemical and biochemical analysis procedures, it is necessary to distribute various reagents and samples precisely and rapidly to multiple wells, and thus microplate-based liquid handling technologies have emerged to meet this demand.

A microplate is a flat plate typically having 6, 12, 24, 96, 384, 1536, 3456 or even 9600 wells arranged in a 2:3 rectangular matrix, in which a small amount of a liquid sample or a liquid reagent may be contained therein. Each well of a microplate holds somewhere between a nanolitres and millilitres volume of the liquid. It is known that such methods include separately adding a reagent and a sample into a same well of a microplate, in which a reaction takes place. A light beam is then applied to the liquid sample, and the intensity of the light passing through the sample is measured to determine the results of the reaction. In this method, the composition of the sample and the content of each component thereof can be determined. Since a very small amount of a sample or a reagent is required in this method, the method is widely employed to examine and diagnose blood or urine, to perform DNA analysis, and other clinical examinations.

Current microplate-based liquid handling methods usually involve processors, detectors, and robotics dispensing mechanisms to deliver reagents and samples to a plurality of wells in microplates, so that reagent-sample reactions or the likes are effectively carried out in the wells. A typical dispensing mechanism is provided with tubes having nozzles. The tube is usually equipped with a pumping device for sucking liquid into the tube and for discharging liquid from the nozzle. The nozzle is cleaned by the sucking and discharging of a clean reagent several times therethrough, in between the delivery of two different liquids. A detachable dispensing tip is often used and mounted to the nozzle, through which liquids can be sucked and discharged therethrough without cross contamination. An additional mechanism is provided for replacing the detachable dispensing tip on the nozzle. The combination of these components in theory allows a large quantity of biochemical tests to be performed simultaneously. The primary technique for saving time and minimizing usage of biological samples involves miniaturization of existing technologies such as low-volume liquid dispensers arranged in parallel and dispensing of the liquids to high-density wells in microplates or microarrays.

Depend upon the relative motion of the dispensing mechanism and the microplate, there are three categories of automatic microplate-based liquid handling system which are disclosed in the prior arts. U.S. Pat. Nos. 7,101,511, 7,169,362 and 7,618,589 describe a microplate-based liquid handling system that has a movable dispensing mechanism operated on a stationary microplate. The automatic microplate liquid handling system is provided with a robotic three-dimensional moving device, a rotating mechanism, a dispensing mechanism, a controller, sensors, an adjustor and a stage. The dispensing mechanism is equipped with a plurality of tubes arranged in a row, and connected to the rotating mechanism and the three-dimensional moving device therewith. The liquid handling system is capable of performing both lateral and longitudinal collective suction/discharge of a liquid on a single microplate. The sensor detects whether the dispensing tip is mounted in the dispensing nozzle. The adjustor aligns the reference positions of the dispensing nozzle and the sensors on an XY plane.

U.S. Pat. Nos. 5,865,224 and 6,044,876 disclose an automatic microplate-based liquid handling system with a stationary dispensing mechanism operated on movable microplates. The stationary dispensing mechanism is equipped with an array of nozzles that dispenses a calibrated quantity of a fluid into a plurality of wells in microplates on a moving stage. The well in the microplate is sequentially moved to the dispensing position so that the corresponding row of wells is aligned with an array of nozzles for dispensing the liquid into the receiving wells.

A third type of automatic microplate-based liquid handling system with a movable dispensing mechanism operated on movable microplates is disclosed in U.S. Pat. Nos. 6,024,925, 6,569,385, 7,232,688, 7,285,422, and 7,390,672. This microplate-based liquid handling system is provided with a computing processer, a motion controller, a robotic arm, a movable dispensing mechanism, a moving stage and movable microplates. The movable dispensing mechanism is connected to the robotic arm, and equipped with an array of pins, wherein each of the pins has an interior chamber and a transducer. The transducer is capable of ejecting liquids from the interior chamber of the pin to a plurality of wells in movable microplates. This microplate-based liquid handling system can perform serial and parallel dispensing of a defined and controlled volume of fluid to generate a multi-element array of sample materials on a substrate surface.

The microplate-based liquid handling systems disclosed in the foregoing patents are designed to meet fixed arrangements of wells in the microplate. The dispensing mechanism is equipped with a row of the liquid dispensers, and delivers liquids to wells in the microplate row by row. However, for a microplate with high density wells, the spacing between two adjacent wells is smaller than the spacing between two adjacent liquid dispensers. Thus, it is difficult to deliver liquids to wells row-by-row in the microplate. Accordingly, the liquid dispensers must be repeatedly aligned to each well, to deliver liquids to the wells. Also, the liquid dispensers must be repeatedly cleaned and refilled in order to avoid the cross contamination problem, when dispensing hundreds or thousands of different liquids in multiple wells.

Detachable dispensing tips are often used and mounted to nozzles, through which liquids can be sucked and discharged therethrough without cross contamination. However, an additional mechanism must be provided to strip the dispensing tip from the nozzle or mount the dispensing tip onto the nozzle. In addition, some amounts of the liquid are likely to adhere to or be deposited on the interior surface of the disposable dispensing tip, which results in inaccurate dispensing volumes.

Current microplate-based liquid handling systems are provided with a microplate reader with one or a limited number of the detectors. One by one the detector detects an optical signal generated from a biological reaction event in each well of the microplate, which slows down the operation of biochemical assay.

Meanwhile, the movable dispensing mechanism is connected to, a robotic system that must make complex two-dimensional or three-dimensional movements to drive the dispensing nozzles to wells in the microplate, which in turn, slows down the operation of biochemical assay. The robotic systems are often burdened by several issues such as high instrumentation costs and a complicated setup and difficult maintenance operations.

### SUMMARY

The disclosure provides a chemical or biochemical analysis apparatus, comprising: a computer processor; at least one controller electrically coupled to the computer processor; at least one first base configured with a plurality of dispensing tube assemblies arranged in a line or alignment and electrically coupled to the at least one controller, independently, wherein each of the plurality of dispensing tube assemblies is electrically coupled to the first base, independently, and is for dispensing a sample, calibrator, control or reagent, independently;at least one second base configured with a plurality of the detectors arranged in a line or alignment and electrically coupled to the at least one controller, wherein each of the plurality of the detectors is electrically coupled to the second base, independently; and a stage, for carrying the at least one multi-well strip having a plurality of wells arranged in a line or alignment and for transporting the multi-well strip to pass through and underneath the plurality of dispensing tube assemblies and the plurality of the detectors in order, electrically coupled to the at least one controller, wherein each well is for receiving at least the sample and the reagent, the calibrator and the reagent, or the control and the reagent, and wherein the detector is used to perform a detection for detecting an event of a chemical or biochemical reaction occurring in the well, and then generating a signal corresponding to the detection and sending the signal to the computer processor.

The disclosure also provides a method for chemical or biochemical analysis, comprising: (a) providing a plurality of dispensing tube assemblies arranged in a line or alignment, for containing and dispensing a sample or reagent, independently; (b) providing a plurality of detectors arranged in a line or alignment; (c) providing at least one multi-well strip having a plurality of wells arranged in a line or alignment; and (d) moving the multi-well strip to pass through and underneath the plurality of dispensing tube assemblies and the plurality of the detectors in order, wherein a selected well of the plurality of wells of the multi-well strip, receives the sample dispensed from a selected dispensing tube assembly containing the sample of the plurality of dispensing tube assemblies and the reagent dispensed from a selected dispensing tube assembly containing the reagent of the plurality of dispensing tube assemblies, and then a selected detector of the plurality of detectors performs a detection for detecting an event of a chemical or biochemical reaction occurring in the well due to the sample and the reagent, and generates a signal corresponding to the detection and send the signal to a computer processor.

The disclosure further provides another method for chemical or biochemical analysis, comprising: (a) providing a plurality of dispensing tube assemblies arranged in a line or alignment, for containing and dispensing a calibrator, control, sample or reagent, independently; (b) providing a plurality of detectors arranged in a line or alignment; (c) providing at least one multi-well strip having a plurality of wells arranged in a line or alignment; and (d) moving the multi-well strip to pass through and underneath the plurality of dispensing tube assemblies and the plurality of the detectors in order, wherein a first selected well of the plurality of wells, receives the calibrator or control dispensed from a selected dispensing tube assembly containing the calibrator or control of the plurality of dispensing tube assemblies and the reagent dispensed from a selected dispensing tube assembly containing the reagent of the plurality of dispensing tube assemblies and a second selected well of the plurality of wells, receives the sample dispensed from a selected dispensing tube assembly containing the sample of the plurality of dispensing tube assemblies and the reagent dispensed from the selected dispensing tube assembly containing the reagent of the plurality of dispensing tube assemblies, and then a selected detector of the plurality of detectors performs a first detection for detecting a first event of a chemical or biochemical reactions occurring in the first well due to the calibrator or control and the reagent and a second detection for detecting a second event of a chemical or biochemical reactions occurring in the second well due to the sample and the reagent, and generates a first signal corresponding to the first detection and a second signal corresponding to the second detection, and send the first and second signals to a computer processor.

A detailed description is given in the following embodiments with reference to the accompanying drawings.

### BRIEF DESCRIPTION OF THE DRAWINGS

The disclosure can be more fully understood by reading the subsequent detailed description and examples with references made to the accompanying drawings, wherein:

FIG. 1 is a schematic view depicting a chemical or biochemical analysis apparatus for dispensing a variety of the liquids to wells in a multi-well strip, and detecting and measuring concentrations of analyts in multiple samples in the disclosure;

FIG. 2A is schematic view depicting a perspective liquid dispensing unit in the disclosure;

FIG. 2B is a drawing of enlargement for dished line marked region 2B in FIG. 2A;

FIG. 3 is a schematic view depicting a perspective dispersing tube assembly in the disclosure;

FIG. 4A is schematic view depicting a perspective detection unit in the disclosure;

FIG. 4B is a drawing of enlargement for dished line marked region 4B in FIG. 4A;

FIG. 5 is a schematic view depicting a perspective multi-well strip in the disclosure;

FIGS. 6A-6D are schematic views depicting an embodiment for a biochemical assaying apparatus of the disclosure;

FIGS. 7A-7C are schematic views depicting an embodiment for a biochemical assaying apparatus of the disclosure;

FIGS. 8A-8B are schematic views depicting an embodiment for a biochemical assaying apparatus of the disclosure;

FIG. 9A is a schematic view depicting a perspective liquid washing unit in the disclosure;

FIG. 9B is a drawing of enlargement for dished line marked region 9B in FIG. 9A;

FIG. 10 is a schematic view depicting a perspective washing tube assembly of the disclosure; and

FIG. 11A-11D show a flowchart illustrating a biochemical assay process conducted by an embodiment of the disclosure

### DETAILED DESCRIPTION OF THE DISCLOSURE

The following description is of the best-contemplated mode of carrying out the disclosure. This description is made for the purpose of illustrating the general principles of the disclosure and should not be taken in a limiting sense. The scope of the disclosure is best determined by reference to the appended claims.

For the various illustrative embodiments, like reference numbers are used to designate like elements.

The words “a”, “an”, and “the” as used herein mean “at least one” unless otherwise specifically indicated.

The term “sample” as used herein refers to a biological liquid specimen that includes one or more analytes with unknown concentrations. The sample may include, and is not limited to, blood, serum, plasma, urine, saliva, sweat or any physiological fluid.

The term “calibrator” as used herein in reference to a biological liquid or solution that includes one or more analytes with known concentrations. A plurality of calibrators are used herein to establish a calibration equation by known concentrations of analytes and resulting signals detected from the chemical or biochemical reaction event in this disclosure.

The term “control” as used herein refers to a biological liquid or solution that includes one or more analytes of known concentrations. The control is used herein to validate the accuracy of a calibration equation by comparing the known concentration of an analyte with the calculated concentration of an analyte from the calibration equation and resulting signals detected from the chemical or biochemical reaction event in this disclosure.

The term “reagent” as used herein refers to a biochemical solution that includes analyte specific reagents as polyclonal or monoclonal antibodies, specific receptors, proteins, ligands, nucleic acid sequences, and similar reagents which, through specific binding or chemical reaction with an analyte in a sample, are intended to be used for diagnostic application for identification and quantification of the analytes in a sample. These analyte specific reagents may bind to nano-particles with or without superparamagnetic properties in the biochemical solution.

In one aspect, the disclosure provide a chemical or biochemical analysis apparatus which is capable of dispensing a variety of predetermined amounts of the liquid samples and reagents independently, into a plurality of wells and performing detection of chemical or biochemical reactions in each of the plurality of wells. In other words, the chemical or biochemical analysis apparatus of the disclosure is capable of performing multiple biochemical analysis procedures which need different amounts or kinds of samples and/or regents during one operation, wherein the chemical or biochemical reactions occurring in each of the multiple biochemical analysis procedures may be the same or different.

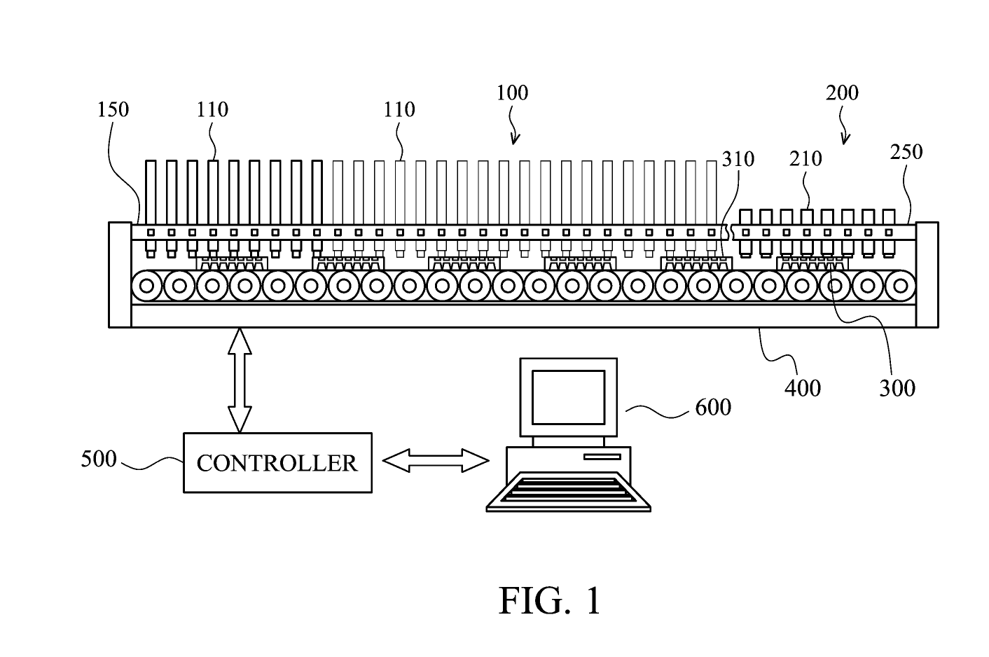

Referring to FIG. 1, in one embodiment, the chemical or biochemical analysis apparatus of the disclosure may comprise a liquid dispensing unit 100, a detector unit 200, a stage 400 for carrying at least one multi-well strip 300, at least one controller 500 and a computer processor 600.

The liquid dispensing unit 100 may comprise at least one first base 150 configured with a plurality of dispensing tube assemblies 110 arranged in a line or alignment and electrically coupled to the at least one controller 500. Each of the plurality of dispensing tube assemblies 110 may be electrically coupled to the first base 150, independently, and be for dispensing a sample, calibrator, control or reagent. Each of the plurality of dispensing tube assemblies 110 may be able to work independently of the other dispensing tube assemblies 110 during operation of the apparatus.

The detector unit 200 may comprise at least one second base 250 configured with a plurality of the detectors 210 arranged in a line or alignment and electrically coupled to the at least one controller 500. Each of the plurality of the detectors 210 may be electrically coupled to the second base 250, independently, and thus each of the plurality of the detectors 210 may be able to work independently of the other detectors 210 during operation of the apparatus.

The at least one multi-well strip 300 carried by the stage 400 may have a plurality of wells 310 arranged in a line or alignment. Each well is able to receive at least the sample and the reagent, the calibrator and the reagent, or the control and the reagent. The stage 400 may transport the multi-well strip 300 passing through and underneath the plurality of dispensing tube assemblies 110 and the plurality of the detectors 210 in order and may be electrically coupled to the at least one controller 500. Moreover, the detector 210 may be used to perform a detection for detecting an event of a chemical or biochemical reaction occurring in the well 310, and then the detector 210 may generate a signal corresponding to the detection and send the signal to the computer processor 600.

For example, when a selected well of the plurality of wells 310 arranged in a line or alignment in the multi-well strip 300, is transported passing under a selected dispensing tube assembly containing a sample of the plurality of dispensing tube assemblies 110, the selected dispensing tube assembly containing the sample will dispense a predetermined amount of sample into the selected well, and when the selected well is further transported passing under a selected dispensing tube assembly containing a reagent of the plurality of dispensing tube assemblies 110, the selected dispensing tube assembly containing the reagent will dispense a predetermined amount of reagent into the selected well. Following, when the selected well is transported passing under a selected detector of the plurality of the detectors 210, the selected detector will perform a detection for detecting an event of a chemical or biochemical reaction occurring in the well 310 due to the sample and the reagent, and then generate a signal corresponding to the detection and send the signal to the computer processor 600.

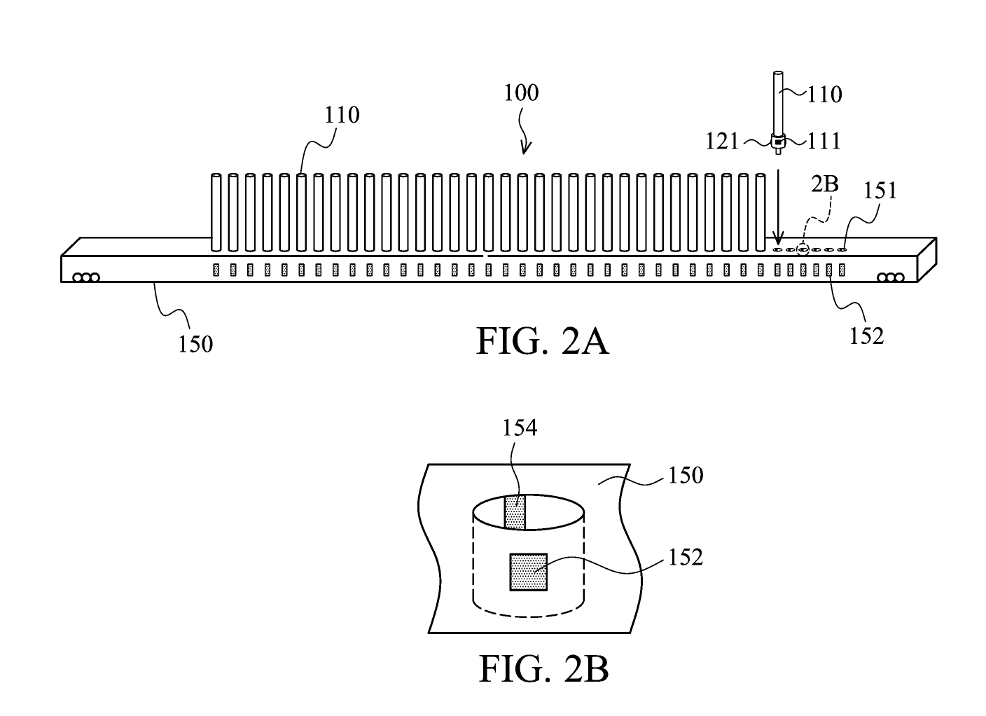

Referring to FIG. 2A, in one embodiment, the first base 150 may have a plurality of holes 151 for configuration of the plurality of dispensing tube assemblies 110. The dispensing tube assembly 110 may be detachable from the first base 150. Also, the dispensing tube assembly 110 may be replaceable. The dispensing tube assembly 110 may be prefilled with the sample, calibrator, control or reagent, but is not limited thereto. In one embodiment, the hole 151 mentioned above may have a first pad 152 on a side wall thereof, and the first pad 152 may be extended into the side wall of the hole 151 and exposed on a surface of the first base 150, wherein the dispensing tube assembly 110 may be electrically coupled to the first base 150 by contact with the first pad 152. FIG. 2B shows a drawing of enlargement for dished line marked region 2B in FIG. 2A.

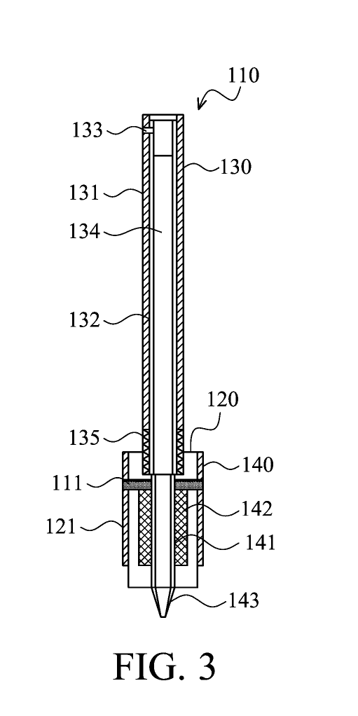

Referring to FIG. 2A and FIG. 3, in one embodiment, the dispensing tube assembly 110 may comprise a receptacle 120, a fluid cartridge 130, and a piezoelectric dispenser 140. The receptacle 120 may comprise a second pad 111 for electrical connection to the first base 150 by contact with the first pad 152 and a passage therein. Referring to FIG. 2B, in one embodiment, the hole 151 of the first base 150 may further comprise at least one recess 154 on the side wall thereof, and the dispensing tube assembly 110 may further comprise at least one protrusion 121 on a side wall of the receptacle 120 corresponding to the at least one recess 154, wherein the protrusion 121 is capable of being inserted into the recess 154. The fluid cartridge 130 may be disposed in the other end of the passage of the receptacle 120. In one embodiment, the fluid cartridge 130 is disposed in the receptacle 120 at the other end of the passage, sealed by a thread 135. The fluid cartridge 130 may comprise a housing 131 having a vent 133 opening to the outer atmosphere and a reservoir tube 132 in the housing 131. The reservoir tube 132 may be for containing or filling in of a fluid 134, such as a sample, calibrator, control or reagent. Moreover, the piezoelectric dispenser 140 may be disposed in the receptacle 120 and may comprise a capillary tube 141, a piezoelectric transducer 142, and a nozzle 143. The capillary tube 141 may be encompassed by and in contact with the piezoelectric transducer 142 and may be connected to the reservoir tube 132. The piezoelectric transducer 142 may be electrically connected to the second pad 111 of the receptacle 120. The nozzle 143 may be connected to the capillary tube 141 and extended out from one end of the receptacle 120. In one embodiment, the piezoelectric transducer 142 is a sleeve of a piezoelectric material coaxial with the capillary tube 141 and has an inner electrode and outer electrode on an inner surface and an outer surface thereof, respectively.

In one embodiment, a high frequency voltage of 1-4000 Hz and 50-300 volts is applied to the inner electrode and the outer electrode of the piezoelectric transducer 142 and causes contractions of the piezoelectric transducer 142, which in turn results in the dispensing of the liquid droplets from the nozzle 143. The piezoelectric transducer 142 is commercially available from several manufacturers such as MicroFab Technologies, Inc. (Plano, Tex., USA) or Vernitron Co. (Laconia, N.H., USA).

In addition, each of the dispensing tube assemblies 110 may be provided with a mark of a first coding indicating the type of fluid contained therein. The first base 150 may be provided with a first detecting device (not shown) for detecting the amount of the fluid. Furthermore, each of the dispensing tube assemblies 110 may be provided with a second detecting device (not shown) for detecting the amount of the fluid remaining in the tube.

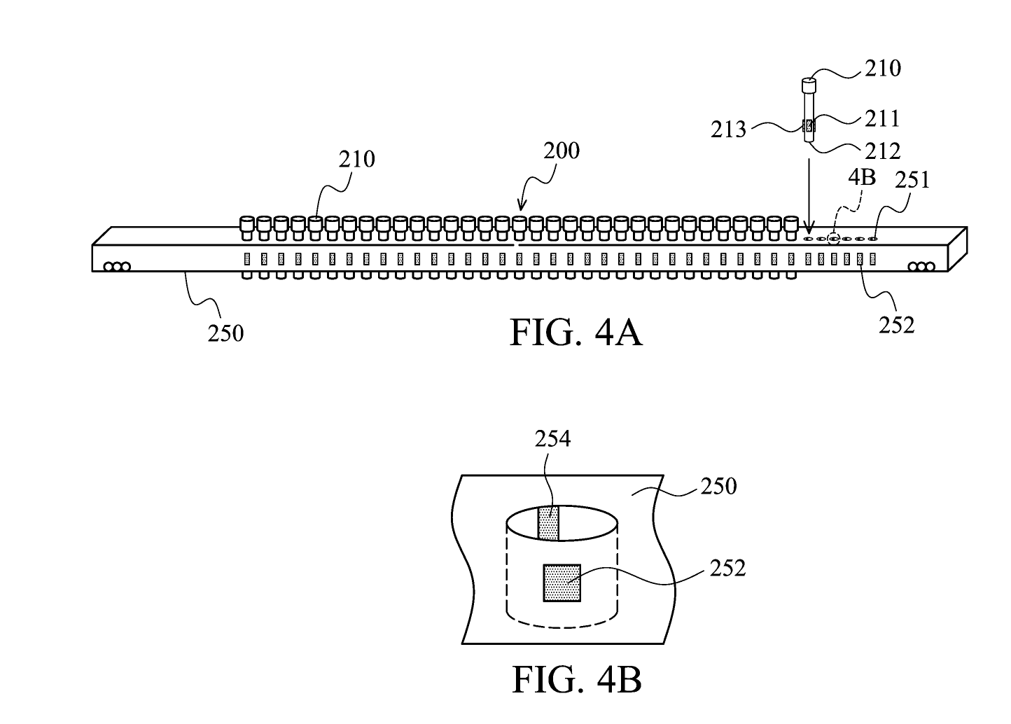

Referring to FIGS. 4A and 4B, in one embodiment, the second base 250 may have a plurality of holes 251 for configuration of the plurality of the detectors 210. The detector 210 may be detachable from the second base 250. Also, the detector 210 may be replaceable. In one embodiment, the hole 251 mentioned above may have a third pad 252 on a side wall thereof, and the third pad 252 may be extended into the side wall of the hole 251 and exposed on a surface of the second base 250, wherein the detector 210 may be electrically coupled to the second base 250 by contact with the second pad 252. FIG. 4B shows a drawing of enlargement for dished line marked region 4B in FIG. 4A.

In one embodiment, the detector 210 may comprise a fourth pad 211 for electrical connection to the second base 250 by contact with the third pad 252 and an optical assembly 212. The optical assembly 212 may comprise a light source for providing a light to the well 310, a light filter, and a light sensor for detecting an optical signal of a specific wavelength generated from the chemical or biochemical reaction event occurring in the well 301. In one embodiment, the hole 251 of the second base 250 may further comprise at least one recess 254 on the side wall thereof, and the detector 210 may further comprise at least one protrusion 213 on a side wall of the optical assembly 212 corresponding to the at least one recess 254, wherein the protrusion 213 is capable of being inserted into the recess 254. Furthermore, each of the detectors 210 is marked with a second coding indicating the type of the detector 210 installed therein.

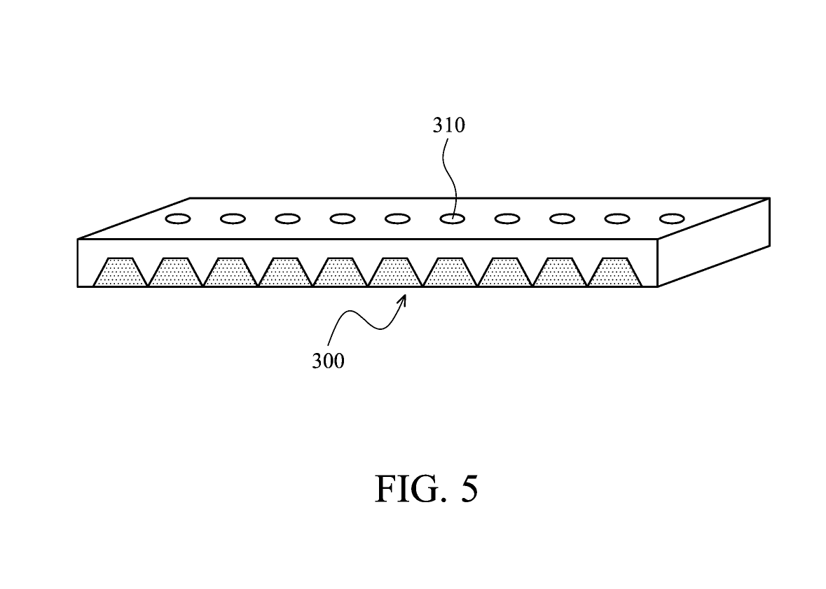

Referring to FIG. 1 and FIG. 5, in one embodiment, the multi-well strip 300 having the plurality of wells 310 arranged in a line or alignment on a substrate is moved underneath the liquid dispensing unit 100 and the detector unit 200 in order by the stage 400. All spacing between the adjacent wells 310 of the multi-well strip 300 is equal and the spacing is large enough for the positioning of the dispensing tube assembly 110 or the detector 210 on top of the well 310. For example, under the control of a controller 500, the liquid dispensing unit 100 separately delivers samples, calibrators, controls and reagents into the wells 310 moving underneath, the detection unit 200 detects the chemical or biochemical reaction events and measures the concentrations of analytes in the samples in the wells 310. In addition, each of the multi-well strips 300 is marked with a third coding indicating its identification number. The distance between the nozzles 143 of the dispensing tube assembly 110 and the well 310 directly underneath, is less than about 1.0-0.1 centimeters, or preferably less than about 0.20 centimeter. The volume of each well 310 in the multi-well strips 300 is less than about 10.0-0.1 microliters, or preferably less than about 1.0 microliter.

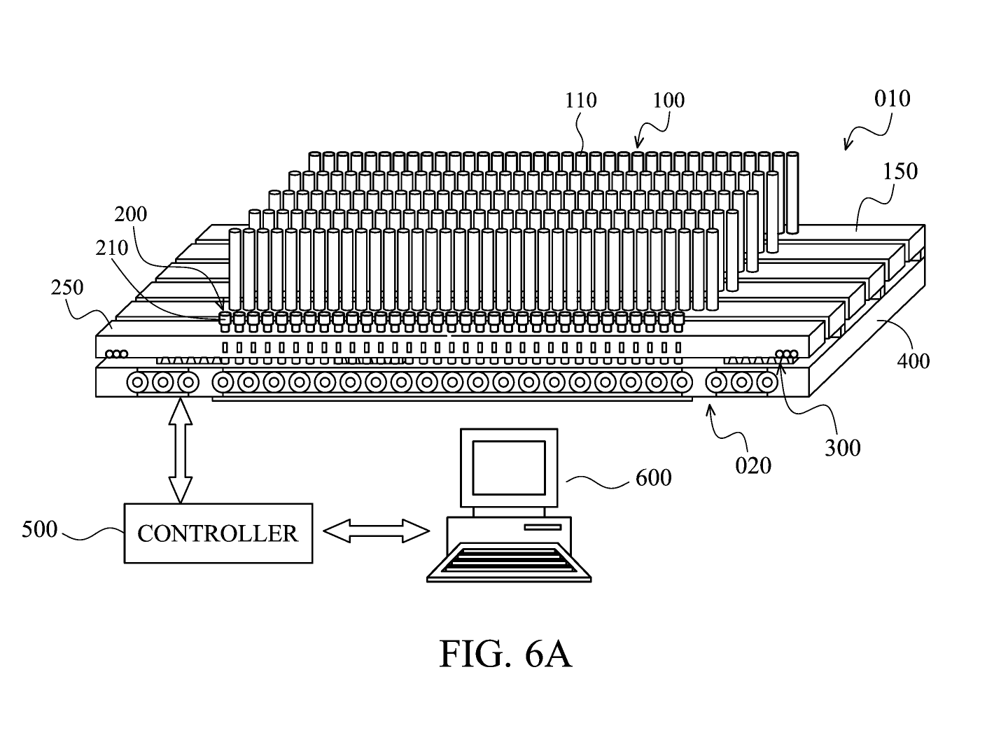

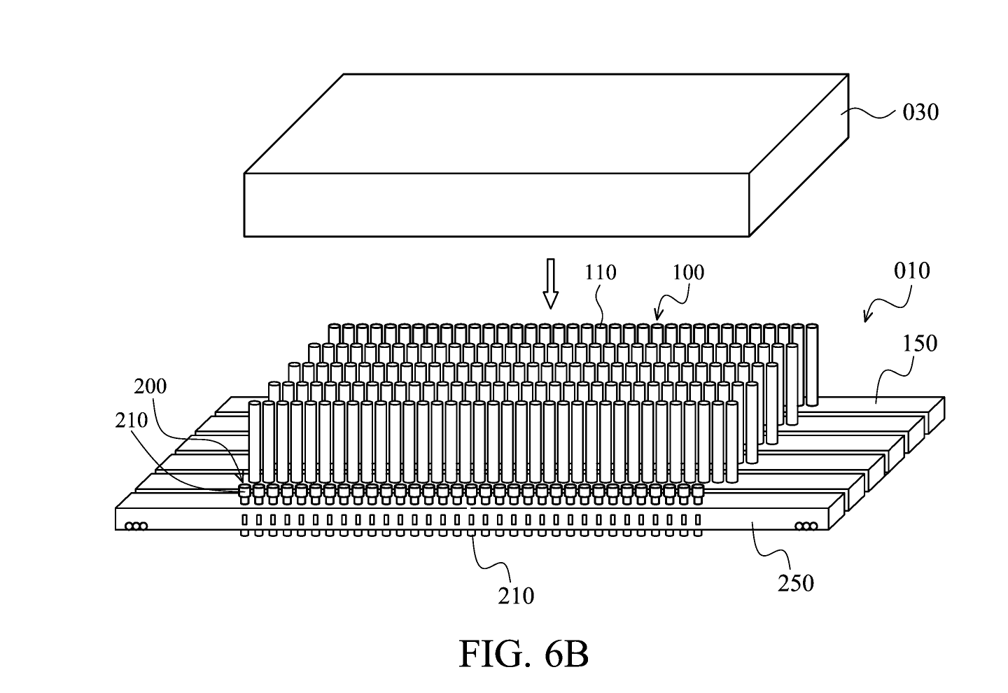

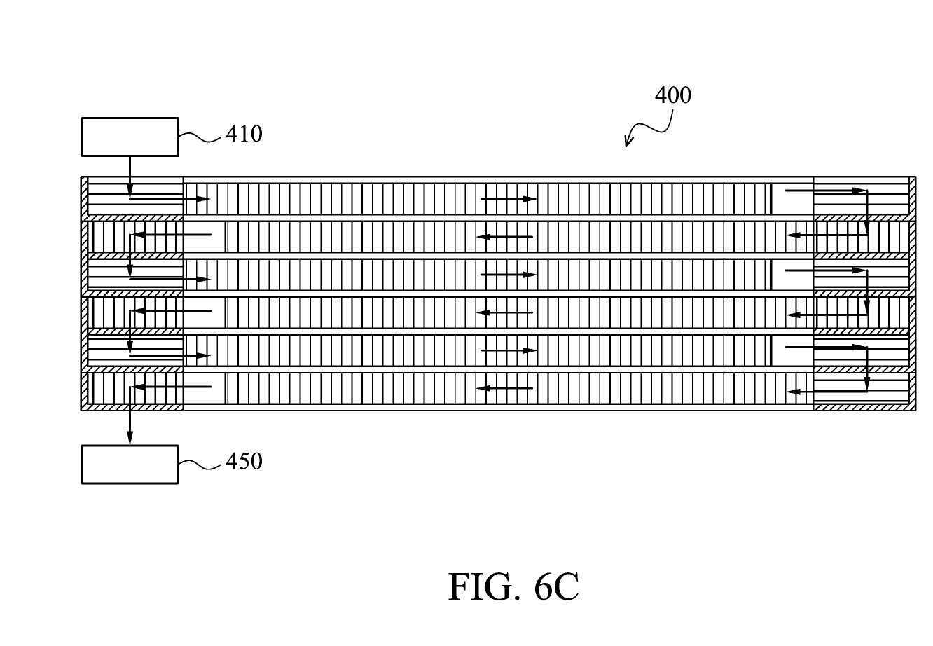

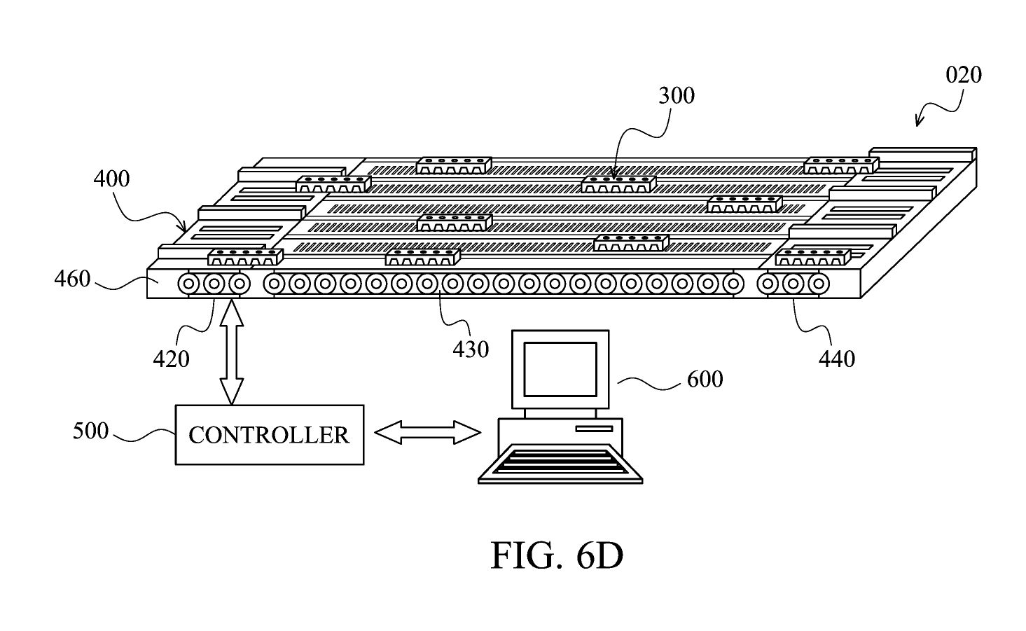

In one embodiment of the apparatus of the disclosure, referring to FIGS. 6A, 6B, 6C and 6D, the at least one liquid dispensing unit 100 (or first base 150 configured with the plurality of dispensing tube assemblies 110 arranged in a line or alignment) mentioned above and the at least one detector unit 200 (or second base 250 configured with the plurality of the detectors 210 arranged in a line or alignment) mentioned above may be arranged in a plurality of parallel lines or alignments to form a top portion 010, and the stage 400 for carrying the at least one multi-well strip 300 is considered as a lower portion 020, as FIG. 6A show. As mentioned above, each liquid dispensing unit 100 is electrically coupled to the controller 500, independently, each detector unit 200 is also electrically coupled to the controller 500, independently, and the controller 500 is electrically coupled to the computer processor 600. The apparatus of this embodiment may further comprise a cooling compartment 030 covering the top portion 010, for cooling the at least one liquid dispensing unit 100 and the at least one detector unit 200, as FIG. 6B show.

FIGS. 6C and 6D show a top view and side view of the stage 400 in the embodiment, respectively. As shown in FIGS. 6C and 6D, the stage 400 may comprise a stage controller 460 electrically coupled to the controller 500, a multi-well strip feeder 410 electrically coupled to the stage controller 460, a first conveyer 420 electrically coupled to the stage controller 460, independently, a second conveyer 430 electrically coupled to the stage controller 460, independently, a third conveyer 440 electrically coupled to the stage controller 460, independently, and a multi-well strip collector 450 electrically coupled to the stage controller 460. The second conveyer 430 is disposed between the first conveyer 420 and the second conveyer 440, and the first conveyer 420 is connected to the second conveyer 430 and the second conveyer 430 is connected to the third conveyer 440. In addition, the second conveyer 430 may have a plurality of parallel belting mechanisms corresponding to the arranged plurality of parallel lines or alignments of the at least one first base 150 and the at least one second base 250 mentioned above, wherein adjacent belting mechanisms are moved in opposite directions. The multi-well strip feeder 410 is capable of feeding the multi-well strip 300 from a stack of the multi-well strips 300 to the first conveyer 420. The first conveyer 420 and the third conveyer 440 are capable of shifting the multi-well strip 300 from a belting mechanism of the plurality of parallel belting mechanisms to an adjacent belting mechanism of the plurality of parallel belting mechanisms in the second conveyer 430, and the multi-well strip 300 may be collected by the multi-well strip collector 450 from the first conveyer 420 or the third conveyer 440. For example, the belting mechanism travels in one direction to a second conveyer 430 carrying the multi-well strip 300 to the third conveyer 440, which in turn, shifts the multi-well strip 300 to another part of the third conveyer 440, which carries the multi-well strip 300 to another belting mechanism of the second conveyer 430 moving in an opposite direction. One by one, each multi-well strip 300 may be moved by the belting mechanisms of the first conveyer 420, the second conveyer 430 and the third conveyer 440. In one embodiment, one by one, each multi-well strip 300 is moved to pass the first conveyer 420, the belting mechanism of the second conveyer 430 and the third conveyer 440, and then finally is collected by the multi-well strip collector 450. In another embodiment, one by one, each multi-well strip 300 is moved to pass the first conveyer 420, the belting mechanism of the second conveyer 430, and the third conveyer 440. After that, each multi-well strip 300 is shifted to an adjacent belting mechanism of the second conveyer 430, and moved to pass the adjacent belting mechanism of the second conveyer 430 and the first conveyer 420, and then is finally collected by the multi-well strip collector 450.

Under the control of the computer processor 600 and the controller 500, one by one, each well 310 in the multi-well strips 300 passes through and underneath each of dispensing tube assemblies 110 in each of the liquid dispensing units 100 and each of the detectors 210 in each of the detection units 200, wherein each of the selected samples, calibrators, controls and reagents is separately delivered to each of the selected wells 310 by each of the selected dispensing tube assemblies 110, and each of the biological reaction events between a reagent and a liquid such as a sample, a calibrator or a control is detected by a selected detector 210, wherein the concentration of an analyte in the selected sample is measured. Selected reagents for detecting corresponding analytes are delivered to selected wells 310 loaded with samples, calibrators or controls by the foregoing described dispensing tube assemblies 110, and react with the liquid therein. Accordingly, selected detectors 210 detect chemical or biochemical reaction events in selected wells in multi-well strips 300 passing through and underneath the detectors 210. Signals generated by the detector from detecting chemical or biochemical reaction events between reagents and calibrators reagents and calibrators or reagents and controls are used to establish calibration curves or quality standards during the chemical or biochemical analysis operation.

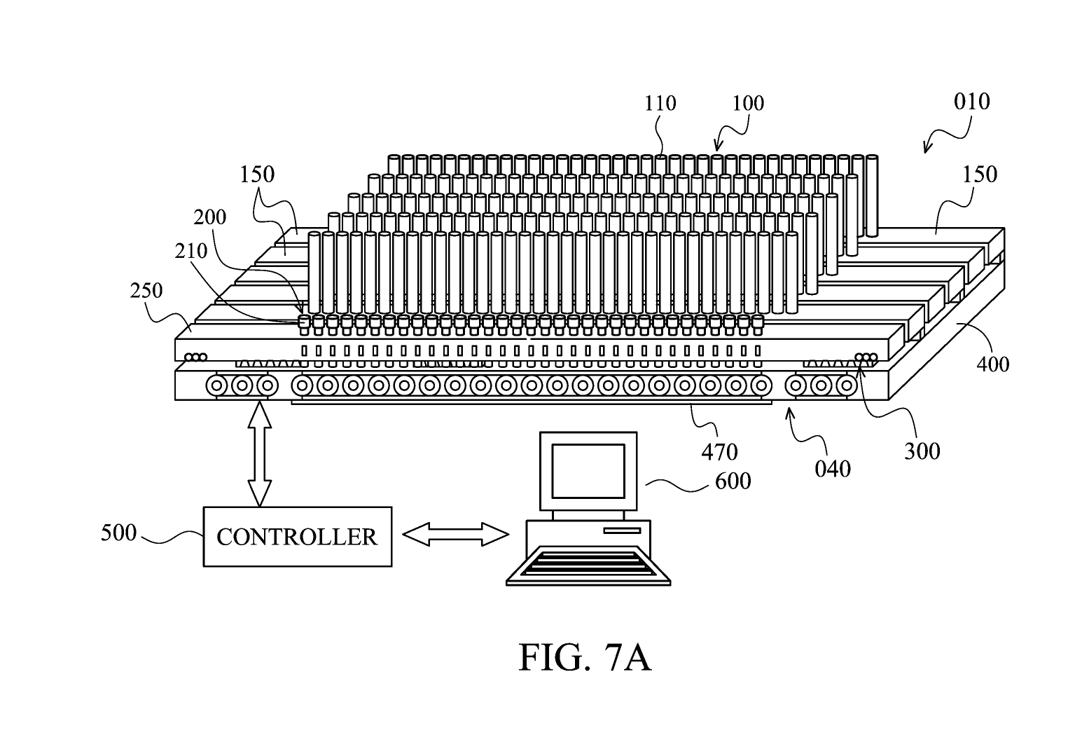

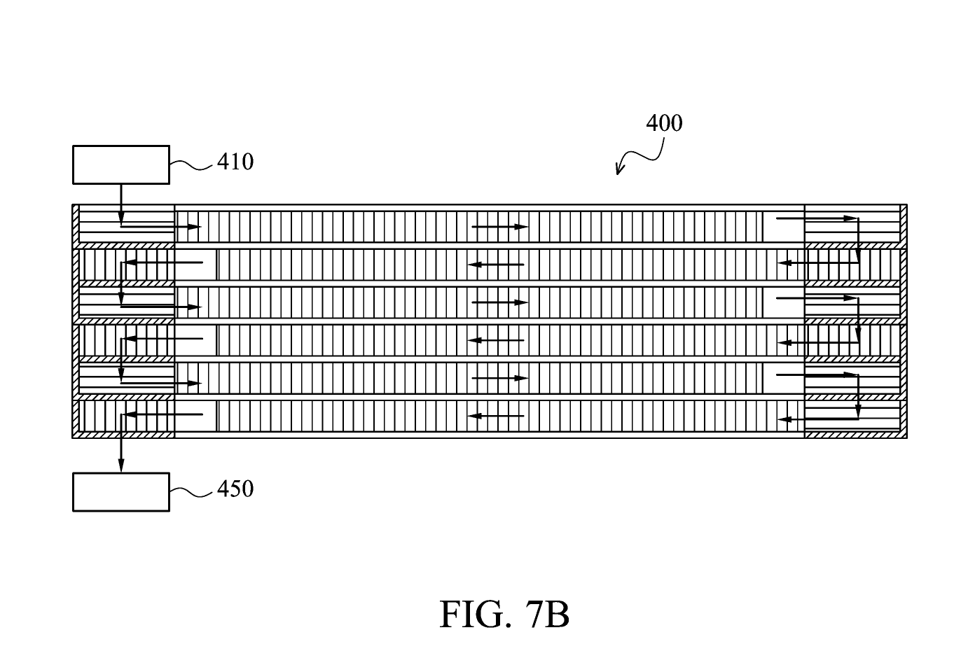

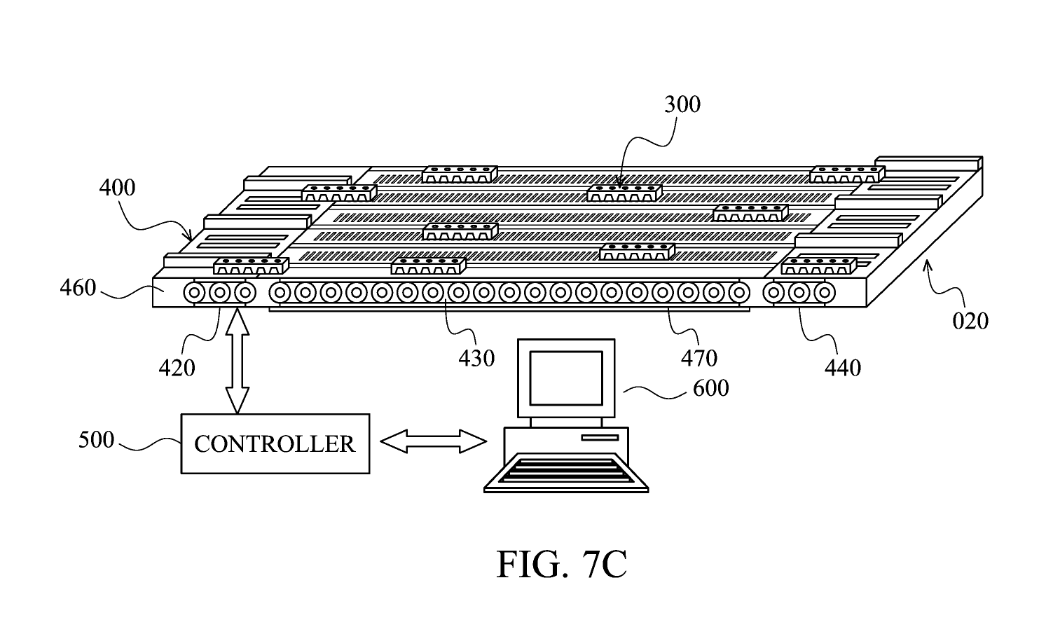

In another embodiment, referring to FIGS. 7A, 7B, 7C and 6B, the at least one liquid dispensing unit 100 (or first base 150 configured with the plurality of dispensing tube assemblies 110 arranged in a line or alignment) mentioned above and the at least one detector unit 200 (or second base 250 configured with the plurality of the detectors 210 arranged in a line or alignment) mentioned above may be arranged in a plurality of parallel lines or alignments to form a top portion 010, and the stage 400 for carrying the at least one multi-well strip 300 is considered as a lower portion 040, as FIG. 7A show. As mentioned above, each liquid dispensing unit 100 is electrically coupled to the controller 500, independently, each detector unit 200 is also electrically coupled to the controller 500, independently, and the controller 500 is electrically coupled to the computer processor 600. The apparatus of this embodiment may further comprise a cooling compartment 030 covering the top portion 010, for cooling the at least one liquid dispensing unit 100 and the at least one detector unit 200, as FIG. 6B show.

FIGS. 7B and 7C show a top view and side view of the stage 400 in the embodiment, respectively. As shown in FIG. 7B and 7C, similar to FIGS. 6C and 6D, the stage 400 may comprise a stage controller 460 electrically coupled to the controller 500, a multi-well strip feeder 410 electrically coupled to the stage controller 460, a first conveyer 420 electrically coupled to the stage controller 460, independently, a second conveyer 430 electrically coupled to the stage controller 460, independently, a third conveyer 440 electrically coupled to the stage controller 460, independently, and a multi-well strip collector 450 electrically coupled to the stage controller 460. The second conveyer 430 is disposed between the first conveyer 420 and the third conveyer 440, and the first conveyer 420 is connected to the second conveyer 430 and the second conveyer 430 is connected to the third conveyer 440. In addition, the second conveyer 430 may have a plurality of parallel belting mechanisms corresponding to the arranged plurality of parallel lines or alignments of the at least one first base 150 and the at least one second base 250 mentioned above, wherein the adjacent belting mechanisms are moved in opposite directions. The multi-well strip feeder 410 is capable of feeding the multi-well strip 300 from a stack of the multi-well strips 300 to the first conveyer 420. The first conveyer 420 and the third conveyer 440 are capable of shifting the multi-well strip from the belting mechanism of the plurality of parallel belting mechanisms to the adjacent belting mechanism of the plurality of parallel belting mechanisms in the second conveyer 430, and the multi-well strip 300 may be collected by the multi-well strip collector 450 from the first conveyer 420 or the third conveyer 440. For example, the belting mechanism travels in one direction to a second conveyer 430 carrying the multi-well strip 300 to a third conveyer 440, which in turn, shifts the multi-well strip 300 to another part of the third conveyer 440, which carries the multi-well strip 300 to another belting mechanism of the second conveyer 430 moving in an opposite direction. One by one, each multi-well strip 300 may be moved by the belting mechanisms of the first conveyer 420, the second conveyer 430 and the third conveyer 440. In one embodiment, one by one, each multi-well strip 300 is moved to pass the first conveyer 420, the belting mechanism of the second conveyer 430 and the third conveyer 440, and then finally is collected by the multi-well strip collector 450. In another embodiment, one by one, each multi-well strip 300 is moved to pass the first conveyer 420, the belting mechanism of the second conveyer 430, and the third conveyer 440. After that, each multi-well strip 300 is shifted to an adjacent belting mechanism of the second conveyer 430, and moved to pass the adjacent belting mechanism of the second conveyer 430 and the first conveyer 420, and then is finally collected by the multi-well strip collector 450.

In addition, in this embodiment, the well 310 of the multi-well strip 300 may be prefilled with at least one metal bead. The metal bead may be coated with a chemical or biochemical substance capable of selectively absorbing a chemical or biochemical molecule in the sample, calibrator, control or reagent. For example, the chemical or biochemical substance may be an antigen, a substrate or a ligand while the chemical or biochemical molecule is an antibody against the antigen, an enzyme specific to the substrate or a receptor for the ligand, or the chemical or biochemical substance may be an antibody, an enzyme or a receptor while the chemical or biochemical molecule may be an antigen for the antibody, a substrate for the enzyme or the ligand for the receptor, but is not limited thereto.

Furthermore, in this embodiment, underneath the at least one detector unit 200 or (second base 250), the belting mechanism is equipped with a plurality of the multi-well strip carriers 470. The multi-well strip carrier 470 may comprise electrical induced magnets and when the electrical induced magnets are electrically induced, the electrical induced magnets generate a magnetic field to implement collection or retaining of the metal bead mentioned above from or on the bottom and/or the side wall of the well 310. When a light beam from the detector 210 is applied to the liquid in the well 310, the intensity of the light passing through the liquid is measured to determine the results of the reaction. Under the control of the stage controller 460, the electric power for the multi-well strip carriers 470 is turned off when the multi-well strip 300 is about to shift from the second conveyer 430 to the first conveyer 420 or the third conveyer 440.

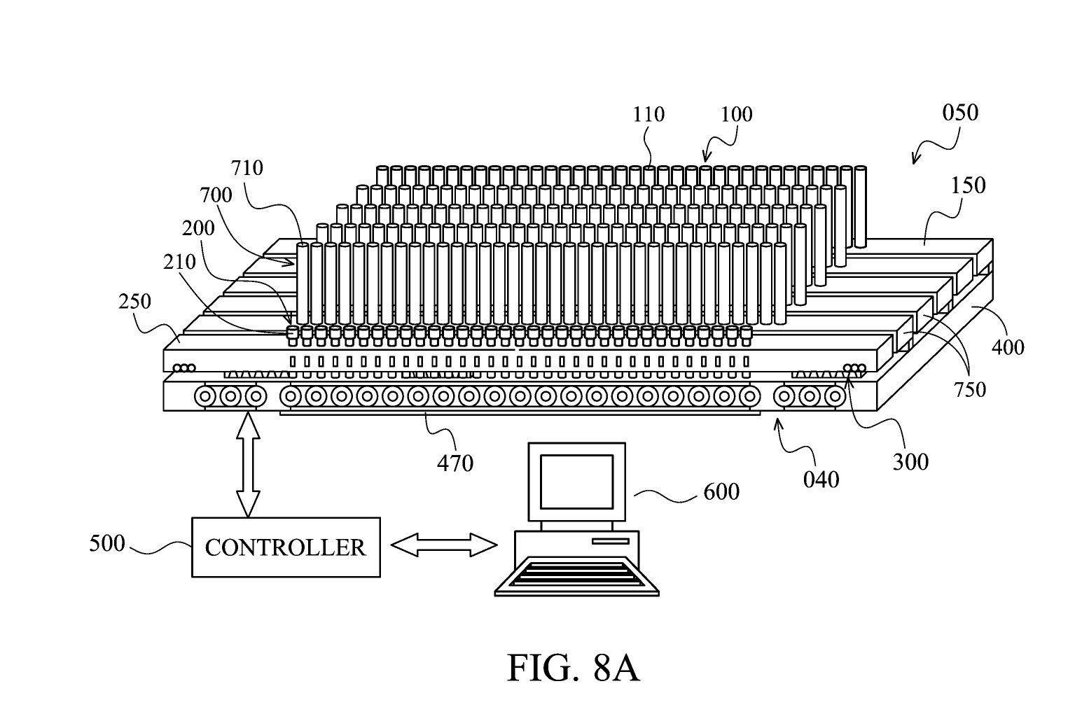

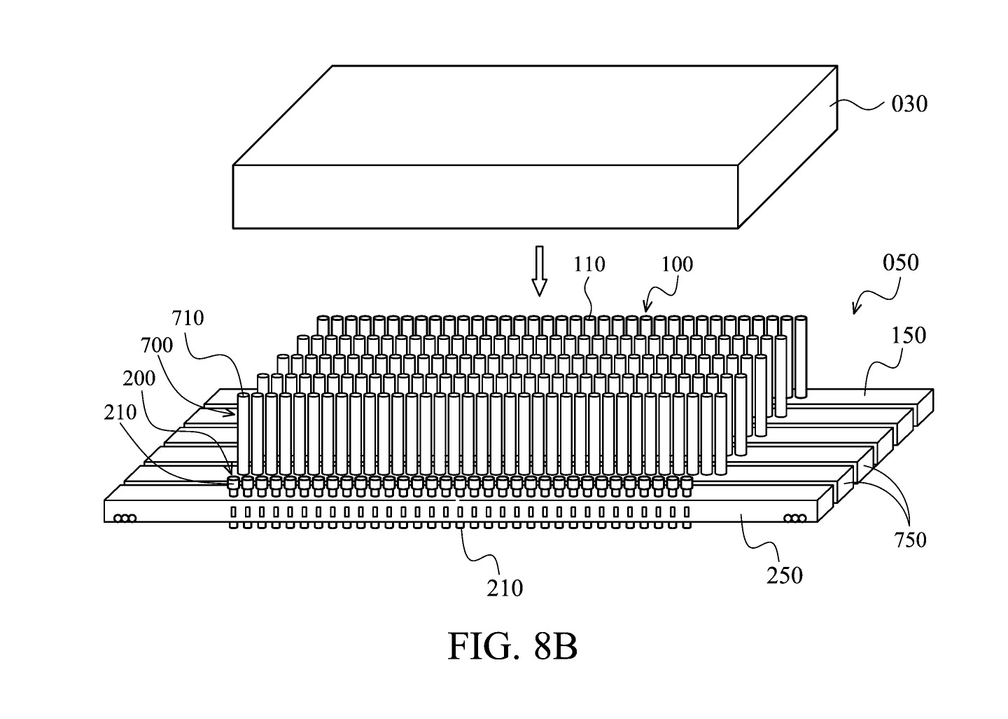

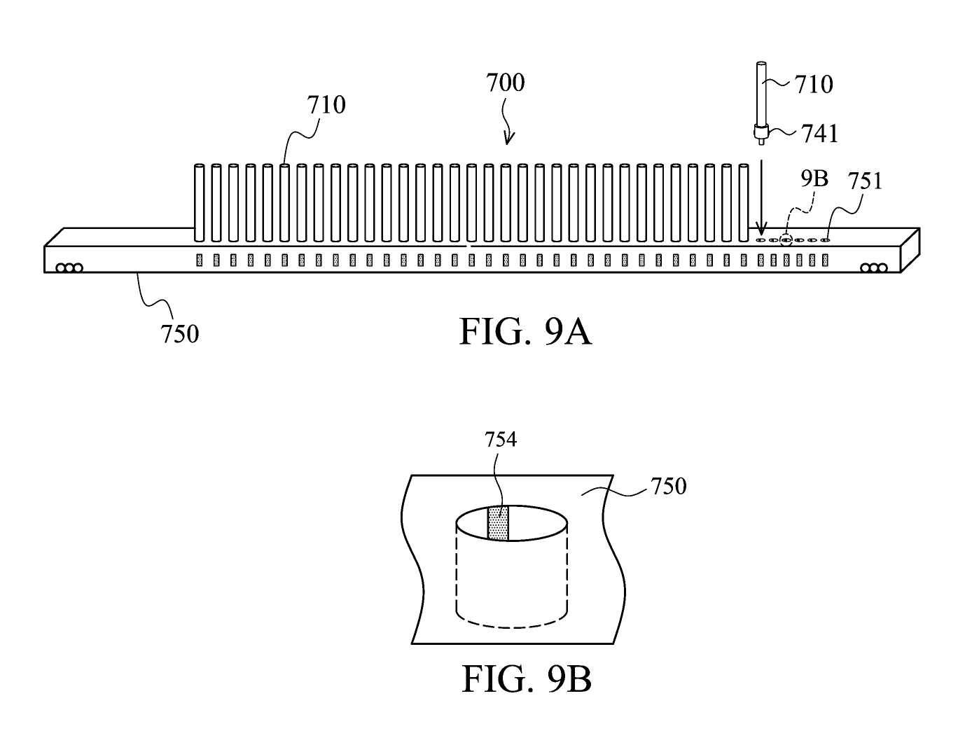

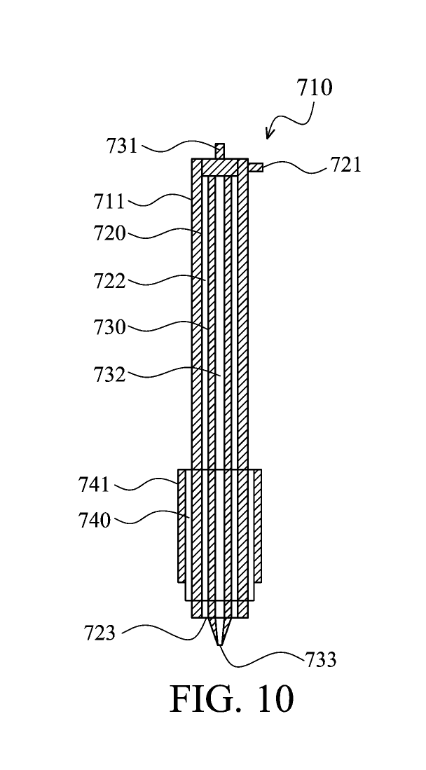

In further another embodiment, referring to FIGS. 8A, 8B, 7B, 7C, 9A and 9B, the at least one liquid dispensing unit 100 (or first base 150 configured with the plurality of dispensing tube assemblies 110 arranged in a line or alignment) mentioned above, at least one liquid wash unit 700 and the at least one detector unit 200 (or second base 250 configured with the plurality of the detectors 210 arranged in a line or alignment) mentioned above may be arranged in a plurality of parallel lines or alignments to form a top portion 050, and the stage 400 for carrying the at least one multi-well strip 300 is considered as a lower portion 040, as FIG. 8A show. Similar to mentioned above, each liquid dispensing unit 100 is electrically coupled to the controller 500, independently, each liquid wash unit 700 is electrically coupled to the controller 500, independently, each detector unit 200 is also electrically coupled to the controller 500, independently, and the controller 500 is electrically coupled to the computer processor 600. The liquid wash unit 700 may comprise a third base 750 configured with a plurality of washing tube assemblies 710 arranged in a line or alignment and electrically connected to the controller 500. Each of the plurality of washing tube assemblies 710 may be electrically connected to the third base 750, independently. In one embodiment, the third base 750 may have a plurality of holes 751 for configuration of the plurality of washing tube assemblies 710, as FIG. 9A show. FIG. 9B shows a drawing of enlargement for dished line marked region 9B in FIG. 9A. The washing tube assembly 710 may be detachable from the third base 750. Also, the washing tube assembly 710 may be replaceable. Furthermore, the apparatus of this embodiment may further comprise a cooling compartment 030 covering the top portion 050, for cooling the at least one liquid dispensing unit 100, at least one liquid wash unit 700 and the at least one detector unit 200, as FIG. 8B show.

In this embodiment, the lower portion 040 of the apparatus of the disclosure is also shown as FIGS. 7B and 7C, similar to FIGS. 6C and 6D. The stage 400 may comprise a stage controller 460 electrically coupled to the controller 500, a multi-well strip feeder 410 electrically coupled to the stage controller 460, a first conveyer 420 electrically coupled to the stage controller 460, independently, a second conveyer 430 electrically coupled to the stage controller 460, independently, a third conveyer 440 electrically coupled to the stage controller 460, independently, and a multi-well strip collector 450 electrically coupled to the stage controller 460. The second conveyer 430 is disposed between the first conveyer 420 and the third conveyer 440, and the first conveyer 420 is connected to the second conveyer 430 and the second conveyer 430 is connected to the third conveyer 440. In addition, the second conveyer 430 may have a plurality of parallel belting mechanisms corresponding to the arranged plurality of parallel lines or alignments of the at least one first base 150, the at least on third base 750 and the at least one second base 250 mentioned above, wherein the adjacent belting mechanisms are moved in opposite directions. The multi-well strip feeder 410 is capable of feeding the multi-well strip 300 from a stack of the multi-well strips 300 to the first conveyer 420. The first conveyer 420 and the third conveyer 440 are capable of shifting the multi-well strip from the belting mechanism of the plurality of parallel belting mechanisms to the adjacent belting mechanism of the plurality of parallel belting mechanisms in the second conveyer 430, and the multi-well strip 300 may be collected by the multi-well strip collector 450 from the first conveyer 420 or the third conveyer 440. For example, the belting mechanism travels in one direction to the second conveyer 430 carrying the multi-well strip 300 to a third conveyer 440, which in turn, shifts the multi-well strip 300 to another part of the third conveyer 440, which carries the multi-well strip 300 to another belting mechanism of the second conveyer 430 moving in an opposite direction. One by one, each multi-well strip 300 may be moved by the belting mechanisms of the first conveyer 420, the second conveyer 430 and the third conveyer 440. In one embodiment, one by one, each multi-well strip 300 is moved to pass the first conveyer 420, the belting mechanism of the second conveyer 430 and the third conveyer 440, and then finally is collected by the multi-well strip collector 450. In another embodiment, one by one, each multi-well strip 300 is moved to pass the first conveyer 420, the belting mechanism of the second conveyer 430, and the third conveyer 440. After that, each multi-well strip 300 is shifted to the adjacent belting mechanism of the second conveyer 430, and moved to pass the adjacent belting mechanism of the second conveyer 430 and the first conveyer 420, and then is finally collected by the multi-well strip collector 450.

In this embodiment, the well 310 of the multi-well strip 300 also may be prefilled with at least one metal bead. The metal bead may be coated with a chemical or biochemical substance capable of selectively absorbing a chemical or biochemical molecule in the sample, calibrator, control or reagent. For example, the chemical or biochemical substance may be an antigen, a substrate or a ligand while the chemical or biochemical molecule is an antibody against the antigen, an enzyme specific to the substrate or a receptor for the ligand, or the chemical or biochemical substance may be an antibody, an enzyme or a receptor while the chemical or biochemical molecule may be an antigen for the antibody, a substrate for the enzyme or the ligand for the receptor, but is not limited thereto.

Furthermore, in this embodiment, underneath the at least one liquid wash unit 700 (or third base 250) and/or the at least one detector unit 200 (or second base 250), the belting mechanism is equipped with a plurality of multi-well strip carriers 470. The multi-well strip carrier 470 may comprise electrical induced magnets and when the electrical induced magnets are electrically induced, the electrical induced magnets generate a magnetic field to implement collection or retaining of the metal bead mentioned above from or on the bottom and/or the side wall of the well 310. When a light beam from the detector 210 is applied to the liquid in the well 310, the intensity of the light passing through the liquid is measured to determine the results of the reaction. Under the control of the stage controller 460, the electric power for the multi-well strip carriers 470 is turned off when the multi-well strip 300 is about to shift from the second conveyer 430 to the first conveyer 420 or the third conveyer 440.

Moreover, referring to FIG. 10, the washing tube assembly 710 may comprise a housing 711, a dispensing tube 720, a dispensing nozzle 723, an aspirating tube 730, an aspirating nozzle 733 and a coaxial receptacle 740. The coaxial receptacle 740 may comprise an inner passage and an outer passage therein. The aspirating tube 730 is disposed in one end of the coaxial receptacle 740 at one end of the inner passage and the aspirating nozzle 733 is disposed at the other end of the inner passage and communicated with the aspirating tube 730. The dispensing tube 720 is disposed in the coaxial receptacle 740 at one end of the outer passage and the dispensing nozzle 723 is disposed at the other end of the outer passage and communicated with the dispensing tube 720. The housing 711 may comprise a liquid inlet 721 and a liquid outlet 731, and cover the dispensing tube 720 and the aspirating tube 730, and the housing 711 may be connected to the other end of the coaxial receptacle 740. The liquid inlet 721 and the liquid outlet 731 are not communicated to each other and the aspirating tube 730 and the dispensing tube 720 are not communicated to each other, while the liquid inlet 721 and the liquid outlet 731 are communicated to the dispensing tube 720 and the aspirating tube 730, respectively. Refer to FIG. 9B, in one embodiment, the hole 751 of the third base 750 may further comprise at least one recess 754 on a side wall thereof and the coaxial receptacle 740 may comprise at least one protrusion 741 corresponding to the at least one recess 754, wherein the protrusion 741 is inserted into the recess 754.

In addition, in one embodiment, each of first bases 150 of the liquid dispensing units 100, each of second bases 250 of the detection units 200 and each of third base 750 of the liquid washing units 700 may be equipped with at least one positioning sensor (not shown) for detecting the relative position between a multi-well strip 300 to a dispensing tube assembly 110, a detector 210 and a washing tube assembly 710, respectively. The data of the relative positions is sent to the computer processor 600 for locating the position of each multi-well strip 300. The position sensor may comprise, but is not limited to, a CCD image sensor or a LED/photo diode sensor.

Under the control of the computer processor 600 and the controller 500, one by one, each well 310 in the multi-well strips 300 passes through and underneath each of dispensing tube assemblies 110 in each of the liquid dispensing units 100 and each of washing tube assemblies 710 in each of the liquid washing units 700 and each of the detectors 210 in each of the detection units 200, wherein each of the selected samples, calibrators, controls and reagents is separately delivered to each of the selected wells 310 by each of the selected dispensing tube assemblies 110, and the liquid mixture in each of the selected wells 310 is removed by a selected washing tube assembly 710, and each of the chemical or biochemical reaction events between a reagent and a liquid such as a sample, a calibrator or a control is detected by a selected detector, wherein the concentration of an analyte in the selected sample is measured, as FIG. 1 and FIG. 9 shows. Selected reagents for detecting corresponding analytes are delivered to selected wells 310 loaded with samples, calibrators or controls by the foregoing described dispensing tube assemblies 110, and react with the liquid therein. Accordingly, selected detectors detect the chemical or biochemical reaction events in selected wells in the multi-well strips passing through and underneath the detectors 210. Signals detected from the chemical or biochemical reaction events between reagents and calibrators or reagents and controls are used to establish calibration curves or quality standards during the biological assaying operation.

In another aspect, the disclosure also provides a method for chemical or biochemical analysis. In one embodiment, the method may comprise the following steps. A plurality of dispensing tube assemblies arranged in a line or alignment, are provided, wherein the plurality of dispensing tube assemblies are used for containing and dispensing a sample or reagent, independently. A plurality of detectors arranged in a line or alignment are provided. At least one multi-well strip having a plurality of wells arranged in a line or alignment, is provided. Then, the multi-well strip is moved to pass through and underneath the plurality of dispensing tube assemblies and the plurality of the detectors in order, wherein a selected well of the plurality of wells of the multi-well strip, receives the sample dispensed from a selected dispensing tube assembly containing the sample of the plurality of dispensing tube assemblies and the reagent dispensed from a selected dispensing tube assembly containing the reagent of the plurality of dispensing tube assemblies, and then a selected detector of the plurality of detectors performs a detection for detecting an event of a chemical or biochemical reaction occurring in the well due to the sample and the reagent, and generates a signal corresponding to the detection and send the signal to a computer processor.

In the embodiment, the method mentioned above may further comprise locating the selected dispensing tube assembly containing the sample, the selected dispensing tube assembly containing the reagent and the selected detector before the multi-well strip is moved to pass through and underneath the plurality of dispensing tube assemblies and the plurality of the detectors in order. In addition, the method may further comprise generating an analysis result for the sample by the computer processor according to the signal after the step the multi-well strip is moved to pass through and underneath the plurality of dispensing tube assemblies and the plurality of the detectors in order.

In another embodiment, the method may comprise the following steps. A plurality of dispensing tube assemblies arranged in a line or alignment, are provided, wherein the plurality of dispensing tube assemblies are used for containing and dispensing a calibrator, control, sample or reagent, independently. A plurality of detectors arranged in a line or alignment are provided. At least one multi-well strip having a plurality of wells arranged in a line or alignment, is provided. Then, the multi-well strip is moved to pass through and underneath the plurality of dispensing tube assemblies and the plurality of the detectors in order, wherein a first selected well of the plurality of wells, receives the calibrator or control dispensed from a selected dispensing tube assembly containing the calibrator or control of the plurality of dispensing tube assemblies and the reagent dispensed from a selected dispensing tube assembly containing the reagent of the plurality of dispensing tube assemblies and a second selected well of the plurality of wells, receives the sample dispensed from a selected dispensing tube assembly containing the sample of the plurality of dispensing tube assemblies and the reagent dispensed from the selected dispensing tube assembly containing the reagent of the plurality of dispensing tube assemblies, and then a selected detector of the plurality of detectors performs a first detection for detecting a first event of a chemical or biochemical reactions occurring in the first well due to the calibrator or control and the reagent and a second detection for detecting a second event of a chemical or biochemical reactions occurring in the second well due to the sample and the reagent, and generates a first signal corresponding to the first detection and a second signal corresponding to the second detection, and send the first and second signals to a computer processor.

In the embodiment, the method may further comprise locating the selected dispensing tube assembly containing the calibrator or control, the selected dispensing tube assembly containing the sample, the selected dispensing tube assembly containing the reagent and the selected detector before the step of the multi-well strip is moved to pass through and underneath the plurality of dispensing tube assemblies and the plurality of the detectors in order. In addition, the method may further comprise generating an analysis result for the sample by the computer processor according to the first and second signals after the step of the multi-well strip is moved to pass through and underneath the plurality of dispensing tube assemblies and the plurality of the detectors in order. Moreover, the analysis result may comprise existence or concentration of an analyte in the sample.

In further another aspect, the disclosure provides a method for chemical or biochemical analysis by using the apparatus of the disclosure mentioned above.

In one embodiment, the method may comprise, but is not limited to, the steps listed below.

At least one dispensing tube assembly of the plurality of dispensing tube assemblies to be prefilled with a sample to be analyzed as the at least one sample dispensing tube assembly is selected and the readiness of the at least one sample dispensing tube assembly is checked by the controller. At least one dispensing tube assembly of the plurality of dispensing tube assemblies to be prefilled with a reagent to be used in the analysis as the at least one reagent dispensing tube assembly is selected and the readiness of the at least one reagent dispensing tube assembly is checked by the controller. At least one detector of the plurality of detectors as the at least one detector to be used to detect during the operation of the analysis is selected and the readiness of the at least one detector is checked by the controller. Then, the at least one sample dispensing tube assembly is located by the controller. The at least one reagent dispensing tube assembly is located by the controller. The at least one detector to be used to detect during the operation of the analysis is located by the controller. After that, moving the at least one multi-well strip to pass through and underneath the plurality of dispensing tube assemblies and the plurality of detectors arranged in order is start via the stage by the controller. At least one well of the at least one multi-well strip to be used to perform analysis is located by the controller. Afterward, the sample is injected into the well from the sample dispensing tube assembly according to the controller if the well is determined to be underneath the sample dispensing tube assembly. The reagent is injected into the well from the reagent dispensing tube assembly according to the controller if the well is determined to be underneath the reagent dispensing tube assembly. Next, a detection for detecting an event of a chemical or biochemical reaction occurring in the well is performed by the detector to be used to detect during the operation of the analysis, if the well is determined to be underneath the detector. A signal corresponding to the detection is generated by the detector. The signal is sent to the computer processor. Finally, an analysis result for the sample is generated by the computer processor according to the signal.

It is noted that, the steps mentioned above may not be performed in order. The sequence for performing the steps of the method may be adjusted, optionally. Moreover, during operation of the apparatus, if conditions are applicable, a plurality of the steps mentioned above may be performed, simultaneously.

In another embodiment, the method may comprise, but is not limited to the steps listed below.

At least one dispensing tube assembly of the plurality of dispensing tube assemblies to be prefilled with a calibrator or control to be used for analysis as the at least one calibrator or control dispensing tube assembly is selected and the readiness of the at least one calibrator or control dispensing tube assembly is checked by the controller. At least one dispensing tube assembly of the plurality of dispensing tube assemblies to be prefilled with a sample to be analyzed as the at least one sample dispensing tube assembly is selected and the readiness of the at least one sample dispensing tube assembly is checked by the controller. At least one dispensing tube assembly of the plurality of dispensing tube assemblies to be prefilled with a reagent to be used in the analysis as at least one reagent dispensing tube assembly is selected and the readiness of the at least one reagent dispensing tube assembly is checked by the controller. At least one detector of the plurality of detectors the at least one detector to be used to detect during the operation of the analysis is selected and the readiness of the at least one detector is checked by the controller. Then, the at least one calibrator or control dispensing tube assembly is located by the controller. The at least one sample dispensing tube assembly is located by the controller. The at least one reagent dispensing tube assembly is located by the controller. The at least one detector to be used to detect during the operation of the analysis is located by the controller. After that moving the at least one multi-well strip to pass through and underneath the plurality of dispensing tube assemblies and the plurality of the detectors arranged in order is started via the stage by the controller. At least two wells of the at least one multi-well strip to be used to perform analysis is located by the controller. Afterward, the calibrator or control is injected into a first well of the at least two wells from the calibrator or control dispensing tube assembly according to the controller if the first well of the at least two wells is determined to be underneath the calibrator or control dispensing tube assembly. The sample is injected into a second well of the at least two wells from the sample dispensing tube assembly according to the controller if the second well of the at least two wells is determined to be underneath the sample dispensing tube assembly. The reagent is injected into the first well of the at least two wells from the reagent dispensing tube assembly according to the controller if the first well of the at least two wells is determined to be underneath the reagent dispensing tube assembly. The reagent is injected into the second well of the at least two wells from the reagent dispensing tube assembly according to the controller if the second well of the at least two wells is determined to be underneath the reagent dispensing tube assembly. Next, a first detection for detecting an event of a chemical or biochemical reaction occurring in the first well of the at least two wells is performed by a first detector of the at least one detector to be used to detect during the operation of the analysis, if the first well is determined to be underneath the first detector. A first signal corresponding to the first detection is generated by the first detector. The first signal is sent to the computer processor. A second detection for detecting an event of a chemical or biochemical reaction occurring in the second well of the at least two wells is performed by a second detector of the at least one detector to be used to detect during the operation of the analysis, if the second well is determined to be underneath the second detector. A second signal corresponding to the second detection is generated by the second detector. The second signal is sent to the computer processor. Analysis information for the calibrator or control and for the sample according to the first signal and the second signal is generated, respectively by the computer processor. Finally an analysis result for the sample is obtained according to the analysis information.

The analysis result may comprise existence or concentration of an analyte in the sample, but is not limited thereto.

It is noted that, the steps mentioned above may not be performed in the order recited above. The sequence for performing the steps of the method may be adjusted, optionally. Moreover, during operation of the apparatus, if conditions are applicable, a plurality of the steps mentioned above may be performed, simultaneously.

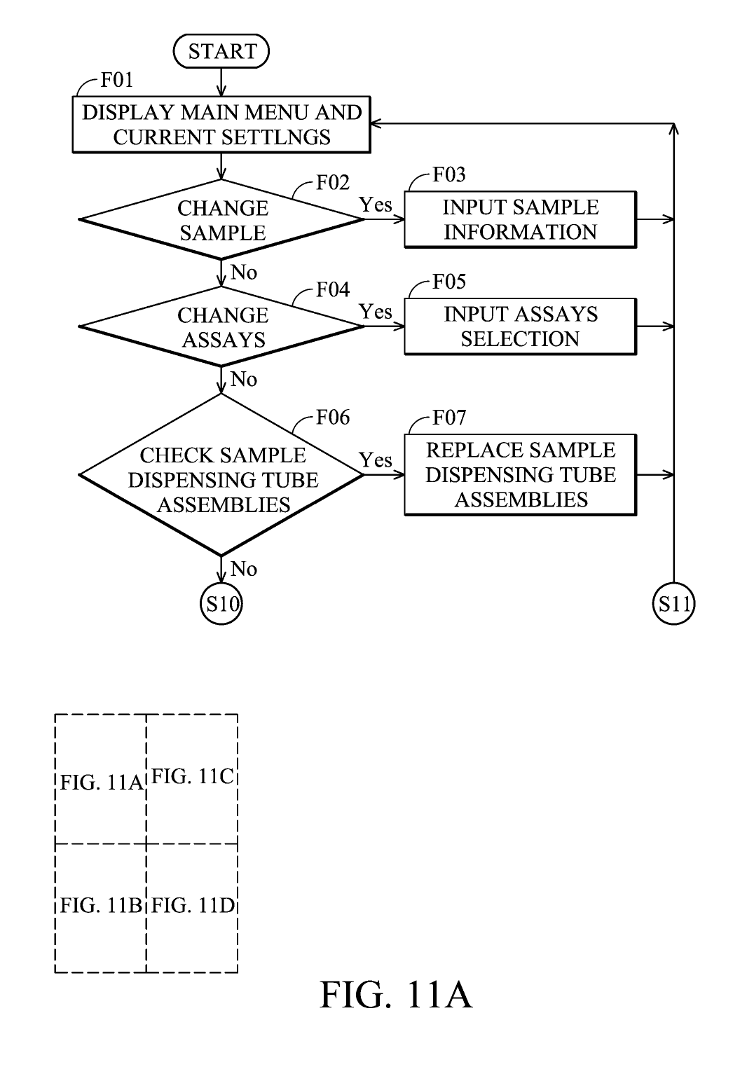

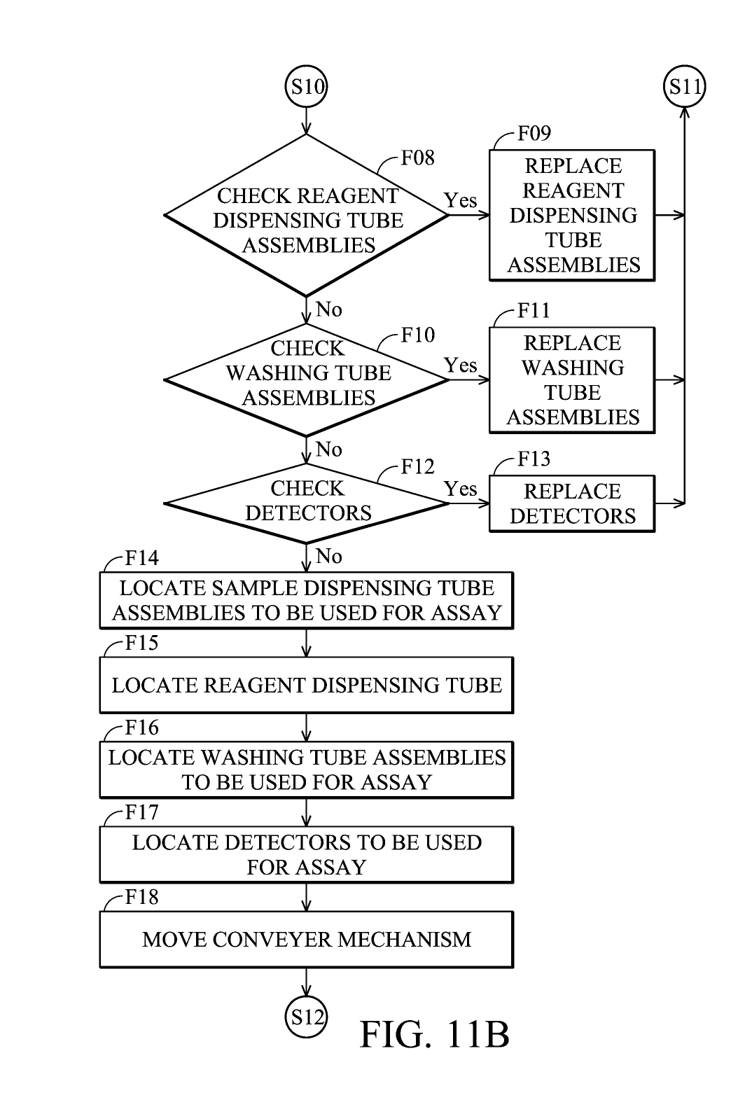

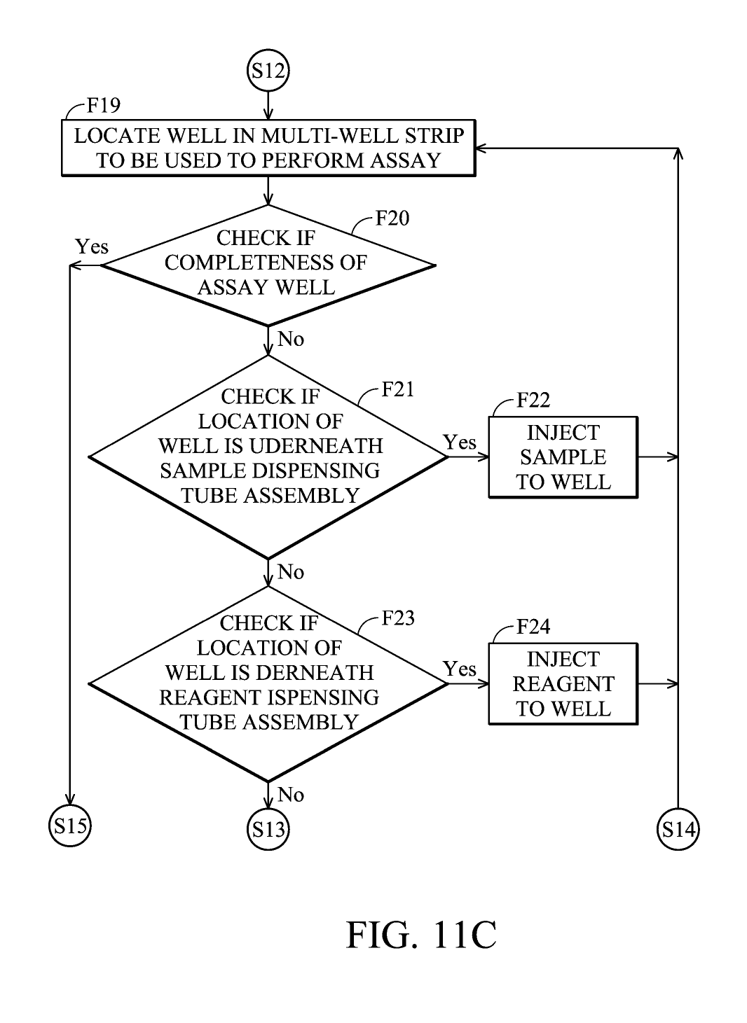

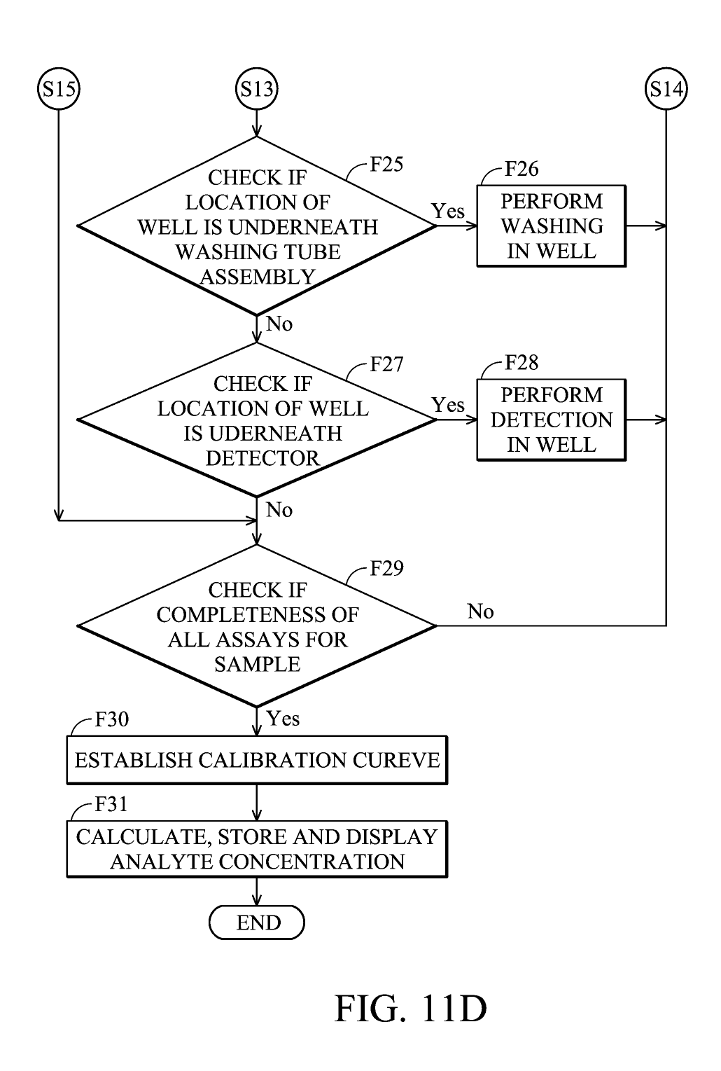

A flowchart for the implementation of chemical or biochemical analysis by the embodiment of the apparatus of the disclosure shown in FIGS. 8A, 8B, 7B and 7C is shown in FIG. 11A to FIG. 11D.

Referring to FIGS. 11A and 11B, in step F01, a main menu and current settings page may be displayed for a user. In one embodiment, the main menu allows a user to change the current settings or to begin a chemical or biochemical assay. The current settings refer to a series of basic information of the chemical or biochemical assay which to be performed, comprising, but is not limited to, the sample information, assay information, and readiness of sample and reagent dispensing tube assemblies 110, washing tube assemblies 710 and detectors 210. After that, in steps F02 and F03, a decision for changing the sample information and a sub-menu page to input the sample information is provided for the user. For example, the sample information may comprise the sample identification number and types of samples, such as blood, serum, plasma, and urine or other physiological fluids, etc. In steps F04 and F05, a decision for changing the assay information and a sub-menu page to input the assay information is provided for the user. The assay information may comprise an assay identification number and the type of analyte in the sample to be analyzed by the assay. In steps F06 and F07, a decision for checking the readiness of the sample dispensing tube assemblies 110 and a sub-menu page for replacing the sample dispensing tube assemblies are provided for the user. Then, in steps F08 and F09, a decision for checking the readiness of the reagent dispensing tube assemblies 110 and a sub-menu page for replacing the reagent dispensing tube assemblies 110 are provided for the user. In step F10 and F11, a decision for checking the readiness of the washing tube assemblies 710 and a sub-menu page for replacing the washing tube assemblies 710 are provided for the user. In steps F12 and F13, a decision for checking the readiness of the detectors 210 and a sub-menu page for replacing the detectors are provided for the user. The checking of the readiness of the sample dispensing tube assemblies 110 can include the verification of the amount of samples stored, the sample identification number, the location of the inserted holes 151 on the first base 150, and the dispensing droplet size and speed. The checking of the readiness of the reagent dispensing tube assemblies 110 may comprise the verification of the amount of reagent stored, the type of reagent, the location of the inserted holes 151 on the first base 150, and the dispensing droplet size and speed. The checking of the readiness of the washing tube assemblies 710 may comprise the location of the inserted holes 751 on third base 750. The checking of the readiness of the detectors 210 can include the location of the inserted holes 251 on the second base 250 and the type of the detector. From the input information of the type of sample and type of analyte in the sample to be analyzed, the computer processor 600 can find an assay procedure from its database and decide the type and amount of reagent and the type of the detector to be used in the assay, wherein sequences of adding the reagents into the samples, and sequences of washing to remove the waste product from the assay mixture are set. In steps F14, F15, F16 and F17, the location of the inserted holes 151 of the sample and reagent dispensing tube assemblies 110, the location of the inserted holes 751 of the washing tube assemblies 710 and the location of the inserted holes 252 of the detectors 210 which to be used for each assay are located. In step F18, the stage 400 starts to carry a plurality of the multi-well strips 300 and moves at a predetermined speed.

Next, as shown in the flowchart of FIGS. 11C and 11D, in step F19, a well 310 in a multi-well strip 300 is selected by the computer processor 600 to perform the assay, and the location of the well 310 in the multi-well strip 300 is also located. In step F21, the computer processor 600 checks if the location of a well 310 in a moving multi-well strip 300 is directly underneath the location of a selected sample dispensing tube assembly 110. If the well 310 is directly underneath the location of a selected sample dispensing tube assemblies 110, in step F22, the computer processor 600 will trigger the selected sample dispensing tube assembly 110 to inject a predetermined amount of sample into the selected well 310. In step F23, the computer processor 600 checks if the location of a well 310 in a moving multi-well strip 300 is directly underneath the location of a selected reagent dispensing tube assembly 110. If a well 310 is directly underneath the selected reagent dispensing tube assembly 110, in step F24, the computer processor 600 will trigger the selected reagent dispensing tube assembly 110 to inject a predetermined amount of sample into the selected well 310. In step F25, the computer processor 600 checks if the location of a well 310 in a moving multi-well strip 300 is directly underneath the location of a selected washing tube assembly 710. If a well 310 is directly underneath the selected washing tube assembly 710, in step F26, the computer processor 600 will trigger the selected multi-well strip carriers 470 to generate magnetic fields to collect and retain the magnetic beads from or on the bottom and/or on the sidewall of the selected well 310 in the multi-well strip 300. The computer processor 600 will also trigger the selected washing tube assemblies 710 to wash away the waste products in the selected well 310. In step F27, the computer processor 600 checks if the location of a well 310 in a moving multi-well strip 300 is directly underneath the location of a selected detector 310. If a well 310 is directly underneath the selected detector 210, in step F28, the computer processor 600 will trigger the selected detector 210 to detect the optical signal in the selected well 310. After each of the steps F22, F24, F26 and F28, the computer processor 600 will update the status of assay reactions in each selected well 310 and will move the procedure to step F20 to re-locate the location of each well 310 in a plurality of the multi-well strips 300 moving on the stage 400. In step F20, the computer processor 600 will check the status of the assay reaction in a selected well 310. If the assay is completed, the computer processor 600 will record the detected optical signal in the selected well 310. In step F29, the computer processor 600 checks if all assays in all of the selected wells 310 have been completely done, otherwise, the computer processor 600 will continue to execute the procedure until all analytes in all samples have been analyzed as requested the user in step F02 and F04. In step F30, the computer processor 600 will use optical signals detected from the assay reaction between the reagents and the calibrators of known concentrations to establish the calibration equations. In step 31, the computer processor 600 will calculate the concentration of analytes in the sample from the detected optical signals and the calibration equations.

While the disclosure has been described by way of example and in terms of the preferred embodiments, it is to be understood that the disclosure is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A chemical or biochemical analysis apparatus, comprising:
a computer processor;
at least one controller electrically coupled to the computer processor;
at least one first base configured with a plurality of dispensing tube assemblies arranged in a line or alignment and electrically coupled to the at least one controller, independently, wherein each of the plurality of dispensing tube assemblies is electrically coupled to the first base, independently, and is for dispensing a sample, calibrator, control or reagent, independently;
at least one second base configured with a plurality of the detectors arranged in a line or alignment and electrically coupled to the at least one controller, wherein each of the plurality of the detectors is electrically coupled to the second base, independently; and
a stage, for carrying the at least one multi-well strip having a plurality of wells arranged in a line or alignment and for transporting the multi-well strip to pass through and underneath the plurality of dispensing tube assemblies and the plurality of the detectors arranged in order, electrically coupled to the at least one controller,
wherein each well is for receiving at least the sample and the reagent, the calibrator and the reagent, or the control and the reagent, and
wherein the detector is used to perform a detection for detecting an event of a chemical or biochemical reaction occurring in the well, and then generating a signal corresponding to the detection and sending the signal to the computer processor.

2. The chemical or biochemical analysis apparatus as claimed in claim 1, wherein the first base comprises a plurality of holes for configuration of the plurality of dispensing tube assemblies.

3. The chemical or biochemical analysis apparatus as claimed in claim 1, wherein the dispensing tube assembly is detachable from the first base.

4. The chemical or biochemical analysis apparatus as claimed in claim 1, wherein the dispensing tube assembly is prefilled with the sample, calibrator, control or reagent.

5. The chemical or biochemical analysis apparatus as claimed in claim 2, wherein the hole has a first pad on a side wall thereof and the first pad is extended into the side wall of the hole and exposed on a surface of the first base, and wherein the dispensing tube assembly is electrically coupled to the first base by contact with the first pad.

6. The chemical or biochemical analysis apparatus as claimed in claim 5, wherein the dispensing tube assembly comprises:
a receptacle comprising a second pad for electrical connection to the first base by contact with the first pad, and a passage therein;
a piezoelectric dispenser in the receptacle, comprising:
a piezoelectric transducer electrically connected to the second pad;
a capillary tube encompassed by and in contact with the piezoelectric transducer; and
a nozzle connected to the capillary tube and extended out from one end of the receptacle; and
a fluid cartridge disposed in the other end of the passage, comprising:
a housing having a vent opening to the outer atmosphere; and
a reservoir in the housing, for containing the sample, calibrator, control or reagent, wherein the reservoir is connected to the capillary tube.

7. The chemical or biochemical analysis apparatus as claimed in claim 6, wherein a distance between the nozzles of the dispensing tube assembly and the well directly underneath, is less than about 1.0 centimeter.

8. The chemical or biochemical analysis apparatus as claimed in claim 7, wherein a distance between the nozzles of the dispensing tube assembly and the well directly underneath, is less than about 0.20 centimeter.

9. The chemical or biochemical analysis apparatus as claimed in claim 1, wherein a volume of each well in the multi-well strips is less than about 10.0 microliter.

10. The chemical or biochemical analysis apparatus as claimed in claim 9, wherein a volume of each well in the multi-well strips is less than about 1.0 microliter.

11. The chemical or biochemical analysis apparatus as claimed in claim 6, wherein the hole further comprises at least one recess on the side wall thereof, and the dispensing tube assembly further comprises at least one protrusion on a side wall of the receptacle corresponding to the at least one recess, and wherein the protrusion is capable of being inserted into the recess.

12. The chemical or biochemical analysis apparatus as claimed in claim 1, wherein the detector is detachable from the second base.

13. The chemical or biochemical analysis apparatus as claimed in claim 1, wherein the second base comprises a plurality of holes for configuration of the plurality of detectors.

14. The chemical or biochemical analysis apparatus as claimed in claim 13, wherein the hole has a third pad on a side wall thereof and the third pad is extended into the side wall of the hole and exposed on a surface of the second base, and wherein the detector is electrically coupled to the second base by contact with the third pad.

15. The chemical or biochemical analysis apparatus as claimed in claim 14, wherein the detector comprises a fourth pad for electrical connection to the second base by contact with the third pad, and an optical assembly.

16. The chemical or biochemical analysis apparatus as claimed in claim 15, wherein the optical assembly comprises:
a light source;
a light filter; and
a light sensor for detecting an optical signal of the specific wavelength generated from the chemical or biochemical reaction event occurring in the well.

17. The chemical or biochemical analysis apparatus as claimed in claim 15, wherein the hole further comprises at least one recess on the side wall thereof, and the detector further comprises at least one protrusion on a side wall of the optical assembly corresponding to the at least one recess, and wherein the protrusion is capable of being inserted into the recess.

18. The chemical or biochemical analysis apparatus as claimed in claim 1, wherein a spacing between the adjacent wells of the multi-well strip is equal, and the spacing is large enough for the positioning of the dispensing tube assembly or the detector on top of the well.

19. The chemical or biochemical analysis apparatus as claimed in claim 1, wherein the at least one first base and the at least one second base are arranged in a plurality of parallel lines or alignments, and the stage comprises:
a stage controller electrically coupled to the controller;
a multi-well strip feeder electrically coupled to the stage controller;
a first conveyer electrically coupled to the stage controller, independently;
a second conveyer having a plurality of parallel belting mechanisms corresponding to the arranged plurality of parallel lines or alignments of the at least one first base and the at least one second base, and electrically coupled to the stage controller, independently, wherein adjacent belting mechanisms are moved in opposite directions;
a third conveyer electrically coupled to the stage controller, independently; and
a multi-well strip collector electrically coupled to the stage controller, wherein the second conveyer is disposed between the first conveyer and the second conveyer, and the first conveyer is connected to the second conveyer and the second is connected to the third conveyer, and
wherein the first conveyer and the second conveyer are capable of shifting the multi-well strip from a belting mechanism of the plurality of parallel belting mechanisms to an adjacent belting mechanism of the plurality of parallel belting mechanisms in the second conveyer, and
wherein the multi-well strip is collected by the multi-well strip collector at the end of the first conveyer or the third conveyer.

20. The chemical or biochemical analysis apparatus as claimed in claim 19, further comprising a cooling compartment for cooling and covering the arranged plurality of parallel lines or alignments of the at least one first base and the at least one second base.

21. The chemical or biochemical analysis apparatus as claimed in claim 19, wherein the well of the multi-well strip comprises at least one metal bead therein, and the metal bead is coated with a chemical or biochemical substance capable of selectively absorbing a chemical or biochemical molecule in the sample, calibrator, control or reagent.

22. The chemical or biochemical analysis apparatus as claimed in claim 21, wherein the chemical or biochemical substance is an antigen while the chemical or biochemical molecule is an antibody against the antigen, or the chemical or biochemical substance is an antibody while the chemical or biochemical molecule is an antigen for the antibody.

23. The chemical or biochemical analysis apparatus as claimed in claim 21, wherein the chemical or biochemical substance is a substrate while the chemical or biochemical molecule is an enzyme specific to the substrate, or the chemical or biochemical substance is an enzyme while the chemical or biochemical molecule is a substrate for the enzyme.

24. The chemical or biochemical analysis apparatus as claimed in claim 21, wherein the chemical or biochemical substance is a ligand while the chemical or biochemical molecule is a receptor specific to the ligand, or the chemical or biochemical substance is a receptor while the chemical or biochemical molecule is a ligand for the receptor.

25. The chemical or biochemical analysis apparatus as claimed in claim 21, wherein underneath the at least one second base, the belting mechanism is equipped with a plurality of multi-well strip carriers comprising electrical induced magnets, and when the electrical induced magnets are electrically induced, the electrical induced magnet generates a magnetic field to collect or retain the metal bead from or on a bottom and/or a side wall of the well.

26. The chemical or biochemical analysis apparatus as claimed in claim 21, further comprising at least one third base configured with a plurality of washing tube assemblies arranged in alignment and electrically connected to the at least one controller, wherein each of the plurality of washing tube assemblies is electrically connected to the third base, independently, and wherein the at least one first base, the at least one third base and the at least one second base are arranged in the plurality of parallel lines or alignments.

27. The chemical or biochemical analysis apparatus as claimed in claim 26, further comprising a cooling compartment for cooling and covering the arranged plurality of parallel lines or alignments of the at least one first base, at least third base and the at least one second base.

28. The chemical or biochemical analysis apparatus as claimed in claim 26, wherein the third base comprises a plurality of holes for configuration of the plurality of washing tube assemblies.

29. The chemical or biochemical analysis apparatus as claimed in claim 26, wherein the washing tube assembly is detachable from the third base.

30. The chemical or biochemical analysis apparatus as claimed in claim 26, wherein the washing tube assembly is replaceable.

31. The chemical or biochemical analysis apparatus as claimed in claim 26, wherein the washing tube assembly comprises:
a coaxial receptacle comprising an inner passage and an outer passage therein;
an aspirating tube disposed in one end of the coaxial receptacle at one end of the inner passage;
an aspirating nozzle disposed at the other end of the inner passage and communicated with the aspirating tube;
a dispensing tube disposed in the coaxial receptacle at one end of the outer passage;
a dispensing nozzle disposed at the other end of the outer passage and communicated with the dispensing tube; and
a housing comprising a liquid inlet and a liquid outlet and covering the dispensing tube and the aspirating tube, connected to the other end of the coaxial receptacle,
wherein the liquid inlet and the liquid outlet are not communicated to each other and the aspirating tube and the dispensing tube are not communicated to each other, while the liquid inlet and the liquid outlet are communicated to the dispensing tube and the aspirating tube, respectively.

32. The chemical or biochemical analysis apparatus as claimed in claim 31, wherein the hole further comprises at least one recess on a side wall thereof and the coaxial receptacle further comprises at least one protrusion corresponding to the at least one recess, and wherein the protrusion inserted into the recess.

33. The chemical or biochemical analysis apparatus as claimed in claim 26, wherein underneath the at least one third base and/or at least one second base, the belting mechanism is equipped with a plurality of multi-well strip carriers comprising electrical induced magnets, and when the electrical induced magnets are electrically induced, the electrical induced magnets generate a magnetic field to collect or retain the metal bead from or on a bottom and/or a side wall of the well.

34. A method for chemical or biochemical analysis, comprising:
(a) providing a plurality of dispensing tube assemblies arranged in a line or alignment, for containing and dispensing a sample or reagent, independently;
(b) providing a plurality of detectors arranged in a line or alignment;
(c) providing at least one multi-well strip having a plurality of wells arranged in a line or alignment; and
(d) moving the multi-well strip to pass through and underneath the plurality of dispensing tube assemblies and the plurality of the detectors in order,
wherein a selected well of the plurality of wells of the multi-well strip, receives the sample dispensed from a selected dispensing tube assembly containing the sample of the plurality of dispensing tube assemblies and the reagent dispensed from a selected dispensing tube assembly containing the reagent of the plurality of dispensing tube assemblies, and then a selected detector of the plurality of detectors performs a detection for detecting an event of a chemical or biochemical reaction occurring in the well due to the sample and the reagent, and generates a signal corresponding to the detection and send the signal to a computer processor.

35. The method for chemical or biochemical analysis as claimed in claim 34, further comprising locating the selected dispensing tube assembly containing the sample, the selected dispensing tube assembly containing the reagent and the selected detector before the step (d).

36. The method for chemical or biochemical analysis as claimed in claim 34, further comprising generating an analysis result for the sample by the computer processor according to the signal after the step (d).

37. A method for chemical or biochemical analysis, comprising:
(a) providing a plurality of dispensing tube assemblies arranged in a line or alignment, for containing and dispensing a calibrator, control, sample or reagent, independently;
(b) providing a plurality of detectors arranged in a line or alignment;
(c) providing at least one multi-well strip having a plurality of wells arranged in a line or alignment; and
(d) moving the multi-well strip to pass through and underneath the plurality of dispensing tube assemblies and the plurality of the detectors in order,
wherein a first selected well of the plurality of wells, receives the calibrator or control dispensed from a selected dispensing tube assembly containing the calibrator or control of the plurality of dispensing tube assemblies and the reagent dispensed from a selected dispensing tube assembly containing the reagent of the plurality of dispensing tube assemblies and a second selected well of the plurality of wells, receives the sample dispensed from a selected dispensing tube assembly containing the sample of the plurality of dispensing tube assemblies and the reagent dispensed from the selected dispensing tube assembly containing the reagent of the plurality of dispensing tube assemblies, and then a selected detector of the plurality of detectors performs a first detection for detecting a first event of a chemical or biochemical reactions occurring in the first well due to the calibrator or control and the reagent and a second detection for detecting a second event of a chemical or biochemical reactions occurring in the second well due to the sample and the reagent, and generates a first signal corresponding to the first detection and a second signal corresponding to the second detection, and send the first and second signals to a computer processor.

38. The method for chemical or biochemical analysis as claimed in claim 37, further comprising locating the selected dispensing tube assembly containing the calibrator or control, the selected dispensing tube assembly containing the sample, the selected dispensing tube assembly containing the reagent and the selected detector before the step (d).

39. The method for chemical or biochemical analysis as claimed in claim 37, further comprising generating an analysis result for the sample by the computer processor according to the first and second signals after the step (d).

40. The method for chemical or biochemical analysis as claimed in claim 37, wherein the analysis result comprises existence or concentration of an analyte in the sample.
